(19) United States
(12) Patent Application Publication
(10) Pub. No.: US 2017/0112620 A1
(43) Pub. Date: Apr. 27, 2017

(54) SYSTEMS AND METHODS OF SEALING A DEPLOYED VALVE COMPONENT

(71) Applicant: Medtronic Vascular, Inc., Santa Rosa, CA (US)

(72) Inventors: Donna Curley, Ballybrit (IE); Frank White, Galway (IE); Marc Anderson, Galway (IE)

(21) Appl. No.: 15/291,909

(22) Filed: Oct. 12, 2016

Related U.S. Application Data
(63) Continuation-in-part of application No. 14/919,771, filed on Oct. 22, 2015.

(51) Int. Cl. A61F 2/24

(52) U.S. Cl. CPC A61F 2/2409; A61F 2220/0016

(57) ABSTRACT

A sealing system for sealing a valve component in a radially expanded deployed configuration includes a valve component and an expandable sealing component. The valve component includes a frame and a prosthetic valve coupled to the frame. The frame defines a central passage with the prosthetic valve disposed therein. The expandable sealing component includes a sealing frame and a skirt coupled to the sealing frame. The expandable sealing component is configured to be inserted in a compressed configuration within the central passage of the valve component with the valve component in the radially expanded deployed configuration. The expandable sealing component is configured to be radially expanded partially within the valve component, with a first portion disposed within the valve component and a second portion disposed longitudinally outside the valve component. The sealing frame may include a first ring and a second ring.

### CROSS-REFERENCE TO RELATED APPLICATIONS

The present application is a continuation-in-part of U.S. patent application Ser. No. 14/919,771 filed Oct. 22, 2015, the contents of which are incorporated by reference herein in their entirety.

### FIELD OF THE INVENTION

The present invention relates to systems and methods for sealing a percutaneously implanted valve component including a prosthetic valve. More particularly, it relates to the systems and methods for sealing a deployed valve component via transcatheter implantation of a sealing component.

### BACKGROUND

Heart valves are sometimes damaged by disease or by aging, resulting in problems with the proper functioning of the valve. Heart valve replacement has become a routine surgical procedure for patients suffering from valve dysfunctions. Traditional open surgery inflicts significant patient trauma and discomfort, requires extensive recuperation times, and may result in life-threatening complications.

To address these concerns, efforts have been made to perform cardiac valve replacements using minimally-invasive techniques. In these methods, laparoscopic instruments are employed to make small openings through the patient's ribs to provide access to the heart. While considerable effort has been devoted to such techniques, widespread acceptance has been limited by the clinician's ability to access only certain regions of the heart using laparoscopic instruments.

Still other efforts have been focused upon percutaneous transcatheter (or transluminal) delivery of replacement cardiac valves to solve the problems presented by traditional open surgery and minimally-invasive surgical methods. In such methods, a valve component including a prosthetic valve is compacted for delivery in a catheter and then advanced, for example through an opening in the native vasculature, and through to the heart, where the valve component is then deployed in a valve annulus (e.g., the aortic valve annulus).

Various types and configurations of prosthetic valves and valve components are available for percutaneous valve replacement procedures. In general, prosthetic valve designs for a heart attempt to replicate the function of the valve being replaced and thus will include valve leaflet-like structures. Prosthetic valves are generally formed by attaching a bioprosthetic valve to a frame made of a wire or a network of wires, creating a valve component. Such valve components can be contracted radially to introduce valve component into the body of the patient percutaneously through a catheter. The valve component can be deployed by radially expanding it once positioned at a desired target site.

In some patients, a wall of the native valve at the target site may be misshapen or heavily calcified. In such cases, the radial expansion of the valve component may not conform to the shape of the wall of the native valve. If the deployed valve component is not 100% coapted to the wall of the native valve, paravalvular leakage (PVL), a serious post surgical complication may arise.

Accordingly, there is a need for a system and method of sealing a valve component to the wall of the native valve after valve component implantation via transcatheter delivery devices and methods.

### SUMMARY OF INVENTION

Embodiments hereof relate to a sealing system for sealing a valve component in a radially expanded deployed configuration to a wall of the native valve. The sealing system includes the valve component and an expandable sealing component. The valve component includes a frame and a prosthetic valve coupled to the frame. The valve component has an inflow portion and outflow portion. The frame defines a central passage with the prosthetic valve disposed therein. The expandable sealing component includes a sealing frame and a skirt coupled to the sealing frame. The expandable sealing component is configured to be inserted in a compressed configuration within the central passage of the valve component with the valve component in the radially expanded deployed configuration. The expandable sealing component is configured to be radially expanded partially within the valve component with a first portion of the expandable sealing component disposed within the valve component and a second portion of the expandable sealing component disposed longitudinally outside the valve component to prevent blood flow radially between the valve component and a wall of the native valve complex.

Embodiments hereof also relate to a method of correcting paravalvular leakage on an installed valvular prosthesis. The valvular prosthesis includes a frame and a prosthetic valve coupled to the frame. The method includes advancing an expandable sealing component in a radially compressed configuration to a location partially within a portion of the frame of the valvular prosthesis with the frame in a radially expanded configuration within a native valve complex. The expandable sealing component includes a sealing frame and a skirt coupled to the sealing frame. The method further includes expanding the expandable sealing component to a radially expanded configuration such that the expandable sealing component prevents blood flow radially between the valvular prosthesis and a wall of the native valve complex.

### BRIEF DESCRIPTION OF DRAWINGS

FIG. 1 is a cutaway side view illustration of a valve component deployed within a native artery.

FIG. 2A is a side perspective illustration of a sealing system according to an embodiment hereof.

FIG. 2B is a bottom view illustration of the sealing system of FIG. 2A.

FIG. 3A is a side perspective illustration of an expandable sealing ring of the sealing system of FIG. 2A.

FIG. 3B is a top or bottom view illustration of the expandable sealing ring of FIG. 3A.

FIG. 3C is a top or bottom view illustration an expandable sealing ring including a sealing material disposed around an outer surface thereof.

FIG. 4A is side perspective illustration of a sealing system according to another embodiment hereof.

FIG. 4B is a top view illustration of the sealing system of FIG. 4A.

FIG. 5A is a side perspective illustration a sealing system according to another embodiment hereof.

FIG. 5B is a perspective view illustration of an alternative expandable sealing ring for use with the sealing system of FIG. 5A.

FIG. 6 is a side perspective illustration a sealing system according to another embodiment hereof.

FIG. 7A is a side perspective illustration a sealing system according to another embodiment hereof.

FIG. 7B is a bottom view illustration of the sealing system of FIG. 7A.

FIG. 8A is a perspective view illustration of an expandable sealing ring of the sealing system of FIG. 7A.

FIG. 8B is a top or bottom view illustration of the expandable sealing ring of FIG. 8A.

FIG. 9 is a top view illustration of an expandable sealing ring according to another embodiment hereof.

FIG. 10A is a side perspective illustration a sealing system according to another embodiment hereof.

FIG. 10B is a top view illustration of the sealing system of FIG. 11A.

FIG. 11 is a top view illustration of an alternative embodiment of the sealing system of FIG. 10A.

FIGS. 12A, 12B, 13A, 13B, 14A, and 14B are simplified illustrations of a method of sealing a valve component in a radially expanded configuration to a wall of a native valve.

FIGS. 15A, 15B, 16A, 16B, 17A, 17B are simplified illustrations of a method of sealing a valve component in a radially expanded configuration to a wall of a native valve according to another embodiment hereof.

FIGS. 18A-18B are simplified illustrations of the method of FIGS. 15A-17B utilizing the sealing ring of FIG. 9.

FIGS. 19A, 19B, 20A, 20B, 21A, and 21B are simplified illustrations of a method of sealing a valve component in a radially expanded configuration to a wall of a native valve using the sealing system of FIGS. 10A-10B.

FIG. 22A is a side perspective illustration of a sealing system according to another embodiment hereof.

FIG. 22B is a side perspective illustration of the expandable sealing component of the sealing system of FIG. 22A.

FIGS. 22C-22E are schematic illustrations of another embodiment of an expandable sealing component.

FIGS. 22F-22G are schematic illustrations of another embodiment of an expandable sealing component.

FIG. 23A is a side illustration of a sealing system according to another embodiment hereof.

FIG. 23B is a side illustration of an expandable sealing component of the sealing system of FIG. 23A.

FIG. 24A is a side perspective illustration a sealing system according to another embodiment hereof.

FIG. 24B is a top view illustration of a first ring of the sealing system of FIG. 24A.

FIG. 24C is a bottom view illustration of a second ring of the sealing system of FIG. 24A.

FIGS. 25A-25D are schematic illustrations of a retrievable expandable sealing component and a method of retrieving the retrievable expandable sealing component.

FIG. 25E is a schematic illustration another embodiment of a retrievable expandable sealing component.

FIGS. 26A, 26B, 26C, 26D, 26E are simplified illustrations of a method of preventing blood flow between a valvular prosthesis and a wall of the native valve complex according to an embodiment hereof.

### DETAILED DESCRIPTION

Specific embodiments of the present invention are now described with reference to the figures, wherein like reference numbers indicate identical or functionally similar elements. The terms “distal” and “proximal”, when used in the following description to refer to a catheter or delivery device, are with respect to a position or direction relative to the treating clinician. Thus, “distal” and “distally” refer to positions distant from, or in a direction away from, the clinician and “proximal” and “proximally” refer to positions near, or in a direction toward, the clinician. When the terms “distal” and “proximal” are used in the following description to refer to a device implanted into a native artery, such as a valve component, they are used with reference to the direction of blood flow from the heart. Thus “distal” and “distally” refer to positions in a downstream direction with respect to the direction of blood flow and “proximal” and “proximally” refer to positions in an upstream direction with respect to the direction of blood flow.

The following detailed description is merely exemplary in nature and is not intended to limit the invention or the application and uses of the invention. Although the description of the invention is in the context of transcatheter aortic valve sealing systems, the invention may also be used in other body passageways where it is deemed useful. Furthermore, there is no intention to be bound by any expressed or implied theory presented in the preceding technical field, background, brief summary, or the following detailed description.

As referred to herein, a valve component used in accordance with and/or as part of the various systems, devices, and methods of the present disclosure may include a wide variety of different configurations, such as a bioprosthetic heart valve having tissue leaflets or a synthetic heart valve having polymeric, metallic, or tissue-engineered leaflets, and can be specifically configured for replacing any heart valve.

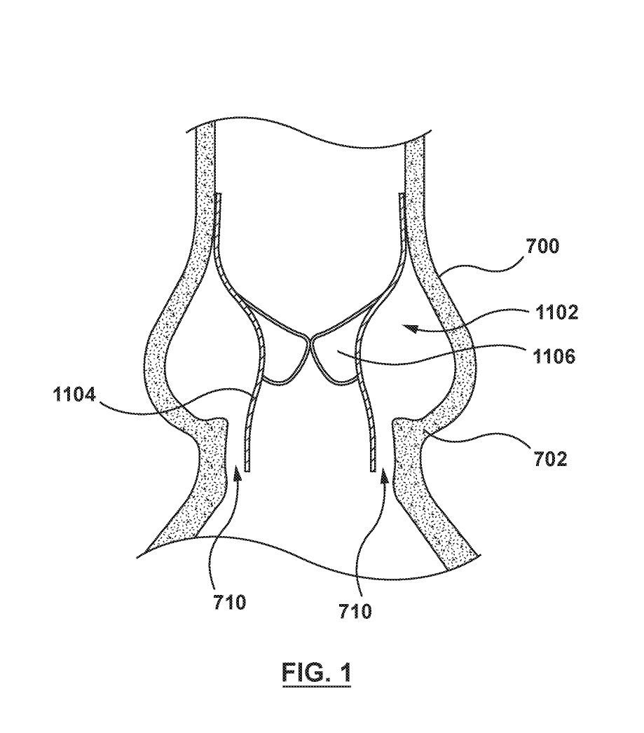

In some patients, the radial expansion of a valve component 1102, including a frame 1104 and a prosthetic valve 1106, as shown in FIG. 1, may not conform to the shape of the wall of the native valve 700. This situation may occur when the wall of the native valve 700 is misshapen or heavily calcified. In such cases where the deployed valve component 1104 is not 100% coapted to the wall of the native valve 700, paravalvular leakage (PVL) may occur between the valve component 1104 and the wall of the native valve. In FIG. 1, voids 710 are shown between the annulus 702 and the valve component 1104. However, this is not meant to be limiting, and such voids may occur between the valve component and the wall of the sinus, or between the valve component and the walls of the sinotubular junction or the ascending aorta. The phrase “wall of the native valve”, as used herein, means the walls surrounding the native valve, including walls slightly downstream and upstream of the native valve. Thus, for example, and not by way of limitation, the “wall of the native valve” for the aortic valve would include the annulus, the wall of the sinuses, the sinotubular junction, and the wall of the ascending aorta.

Embodiments hereof are related to a sealing system including a valve component and an expandable sealing ring. The term valve component, described in more detail below, may also be referred to as a valve prosthesis or valvular prosthesis, or other terms known to those skilled in the art.

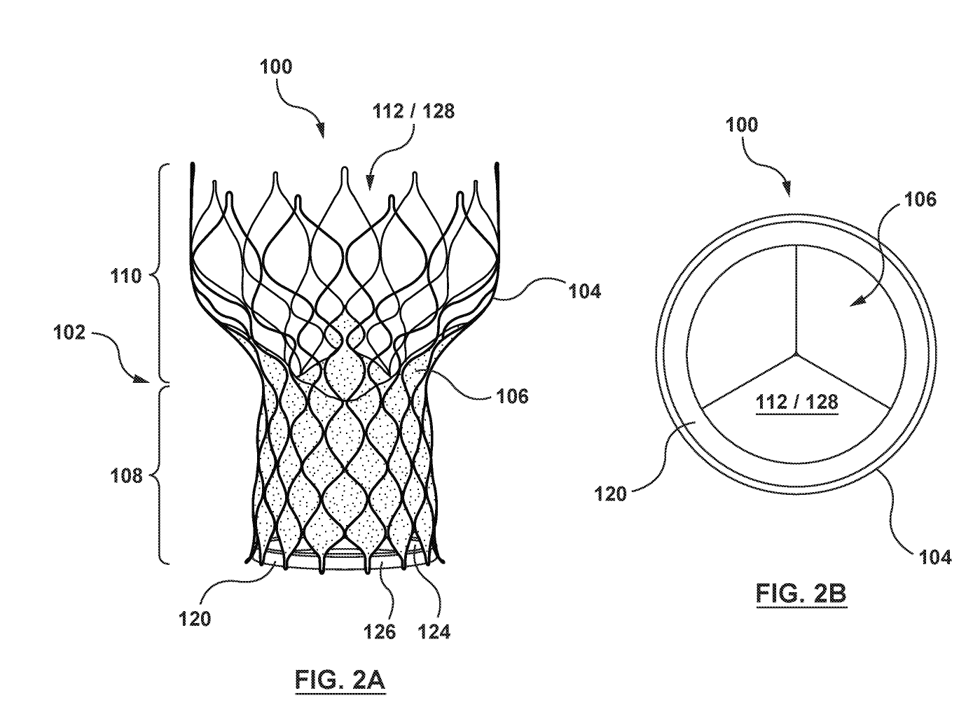

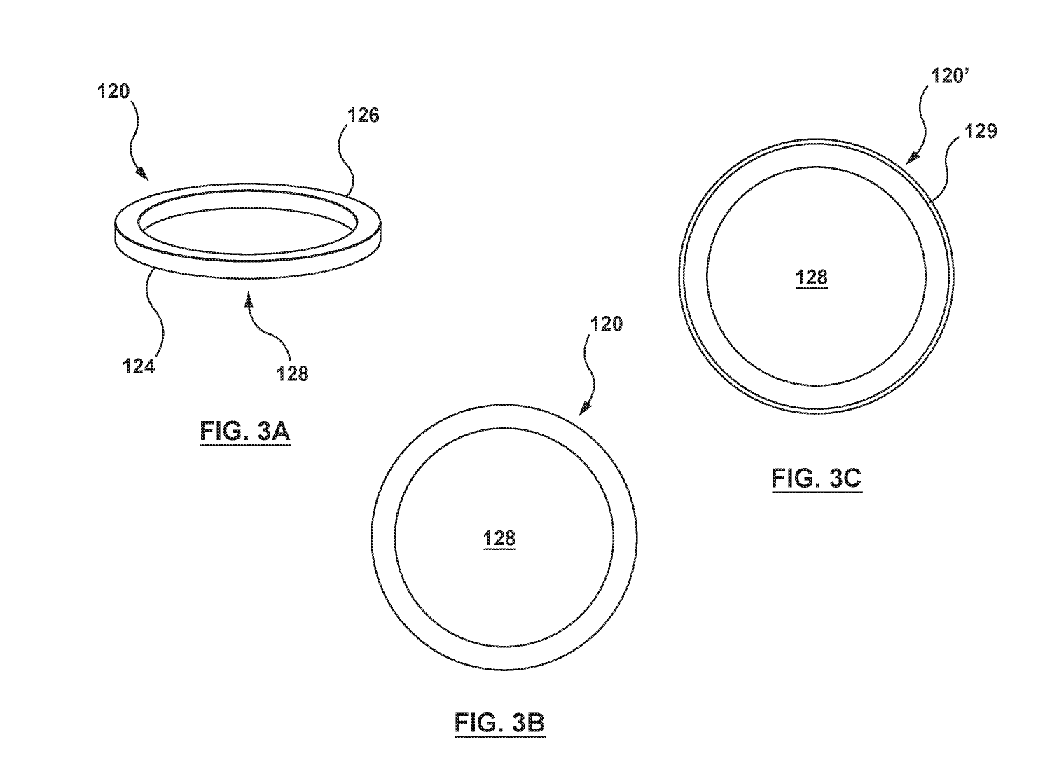

In an embodiment shown in FIGS. 2A, 2B, 3A, and 3B, a sealing system 100 includes a valve component 102 and an expandable sealing ring 120. Sealing system 100 may also be referred to as a device for remodeling a valvular prosthesis. In FIG. 2A, valve component 102 is in a radially expanded configuration.

Valve component 102 includes a frame 104 and a prosthetic valve 106. Valve component 102 may be a conventional valve prosthesis similar to the Medtronic CoreValve® transcatheter aortic valve replacement valve prosthesis and as described in U.S. Pat. No. 7,914,569 to Nguyen et al. (hereinafter “the '569 patent”), which is incorporated by reference herein in its entirety.

Frame 104 is a support structure that comprises a number of struts or wire portions arranged relative to each other to provide a desired compressibility and strength to prosthetic valve 106. Frame 104 is a stent structure as is known in the art. Frame 104 may be self-expandable, balloon-expandable, or otherwise mechanically expandable. Frame 104 may be any stent structure suitable for use with a prosthetic valve. For example, and not by way of limitation, frame 104 may be similar to the stent structures described in the '569 patent and U.S. Pat. No. 7,740,655 to Birdsall, which is incorporated by reference herein. Frame 104 is a generally tubular structure and defines a central passage 112.

Prosthetic valve 106 is coupled to and disposed within frame 104. Prosthetic valve 106 preferably includes individual leaflets formed from a natural or man-made material, including but not limited to, mammalian tissue, such as porcine, equine or bovine pericardium, or a synthetic or polymeric material. Prosthetic valve 106 may also include a skirt (not shown) affixed to frame 104, the leaflets of prosthetic valve 106 may be attached are attached along their bases to the skirt, for example, using sutures or a suitable biocompatible adhesive. Adjoining pairs of the leaflets are attached to one another at their lateral ends to form commissures (not shown), with free edges of the leaflets forming coaptation edges that meet in an area of coaptation, as described in the '569 patent.

Valve component 102 has an inflow portion 108 at a proximal end of valve component 102, and an outflow portion 110 at a distal end of valve component 102, as shown in FIG. 2A.

Expandable sealing ring 120 is a generally annular ring having a longitudinal first end 124 and a longitudinal second end 126 opposite first end 124, as shown in FIGS. 3A and 3B. Expandable sealing ring 120 has a compressed configuration for delivery to a treatment site and an expanded configuration when deployed. Expandable sealing ring 120 may be self-expanding, balloon expandable, or otherwise mechanically expandable. In the expanded configuration, expandable sealing ring 120 may have a diameter in the range of 18 to 29 millimeters for use in an aortic annulus. However, it is recognized that expandable sealing ring 120 may have a smaller or larger expanded diameter depending on the application. Further, the unrestrained expanded diameter of expandable sealing ring 120 is generally about 2-6 millimeters larger than the diameter of the location in which expandable sealing ring 120 is to be installed, in order to create opposing radial forces between the outward radial force of expandable sealing ring 120 against inward resisting forces of the wall of the native valve. Expandable sealing ring 120 may be constructed of materials such as, but not limited to stainless steel, Nitinol, cobalt-chromium alloys (e.g., L605), nickel-cobalt-chromium alloys (e.g., MP35N®) or other materials suitable for the purposes described herein. Expandable sealing ring 120 defines a passage 128, as shown in FIG. 3B. In another embodiment, shown in FIG. 3C, an expandable sealing ring 120′ may include a sealing material 129 disposed around an outer surface thereof. Sealing material 129 may be made of materials such as, but not limited to, nylon, polybutester, silk, polyester, flexible and impermeable materials such as PTFE, and other materials suitable for the purposes described herein. Further, other embodiments described below may also include such sealing materials.

In the embodiment of FIGS. 2A-2B, expandable sealing ring 120 is deployed at inflow portion 108 of valve component 102. In the embodiment of FIGS. 2A-2B, expandable sealing ring 120 is also disposed entirely within valve component 102, such that first end 124 and second end 126 are disposed within valve component 102. Although not shown, a single expandable sealing ring 120 may alternatively be disposed entirely within outflow portion 110 of valve component 102.

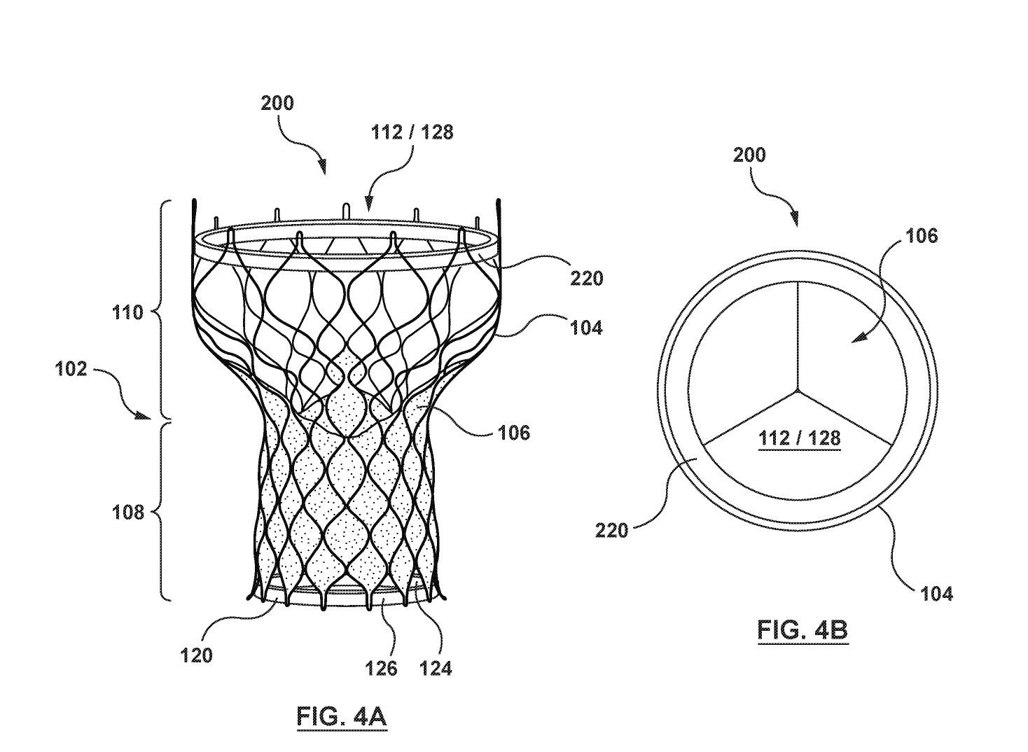

FIGS. 4A-4B show a sealing system 200 according to another embodiment hereof. Sealing system 200 includes a valve component 102 including a frame 104 and a prosthetic valve 106, as described above with respect to FIGS. 2A-2B. Sealing system 200 also includes an expandable sealing ring 120 disposed entirely within inflow portion 108 of valve component 102, as described above and shown in FIG. 2A. Sealing system further includes a second expandable sealing ring 220 disposed entirely within outflow portion 108 of valve component 102. Sealing system 200 uses the same reference numerals as sealing system 100 of FIGS. 2A-2B for items that are similar or identical to the embodiment of FIGS. 2A-2B.

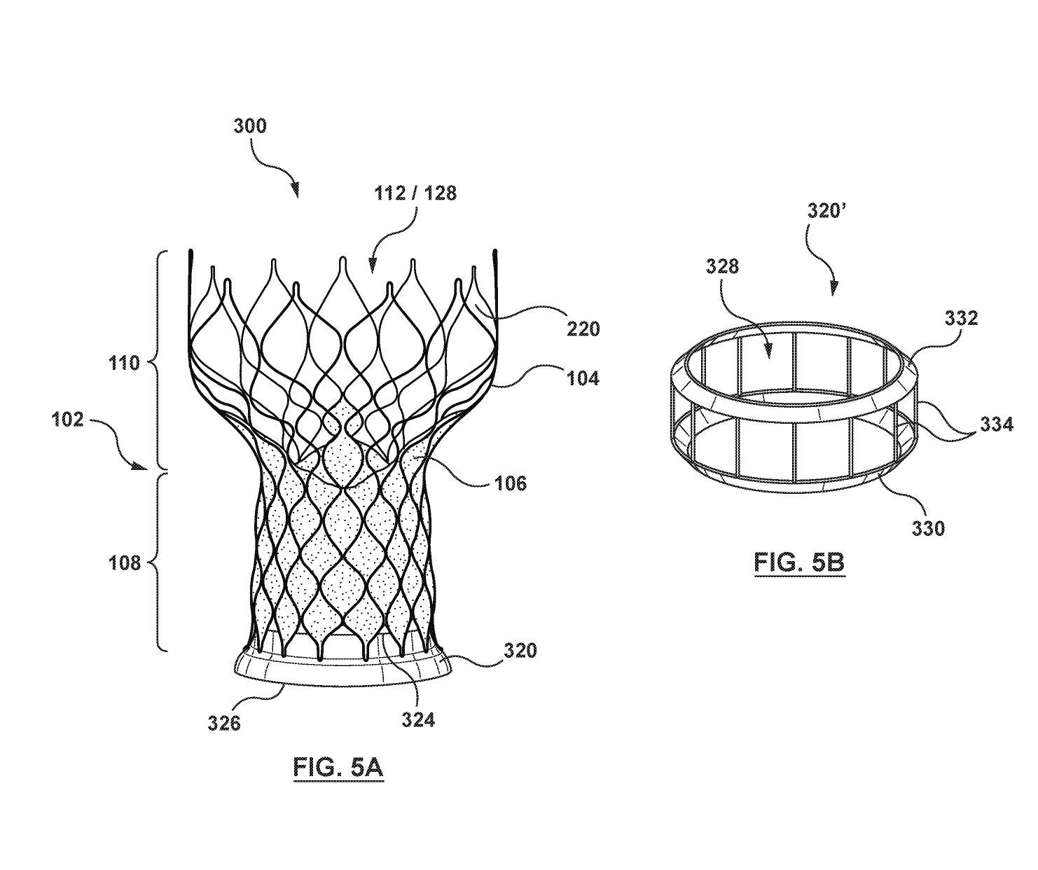

FIG. 5A show a sealing system 300 according to another embodiment hereof. Sealing system 300 includes a valve component 102 including a frame 104 and a prosthetic valve 106, as described above with respect to FIGS. 2A-2B. Sealing system 300 also includes an expandable sealing ring 320 partially disposed within valve component 102 such that a longitudinal first end 324 of expandable sealing ring 320 is disposed longitudinally outside of valve component 102 and longitudinal second end 326 of expandable sealing ring 320 is disposed longitudinally within valve component 102. In the embodiment shown FIG. 5A, expandable sealing ring 320 is deployed at inflow portion 108 of valve component 102. Expandable sealing ring 320 alternatively may be partially disposed at outflow portion 110 of valve component 102 such that first end 324 of expandable sealing ring 320 is disposed within valve component 102 and second end 326 is disposed longitudinally outside of valve component 102.

In an embodiment, expandable sealing ring 320 may simply be longitudinally longer than sealing ring 120 described above, as shown in FIG. 5A. In another embodiment shown in FIG. 5B, expandable sealing ring 320′ includes a first ring 330, a second ring 332, and longitudinal connectors 334 coupling first ring 330 and second ring 332 to each other. In such an embodiment, first ring 330 may be disposed longitudinally outside of valve component 102 and second ring 332 may be disposed longitudinally within valve component 102.

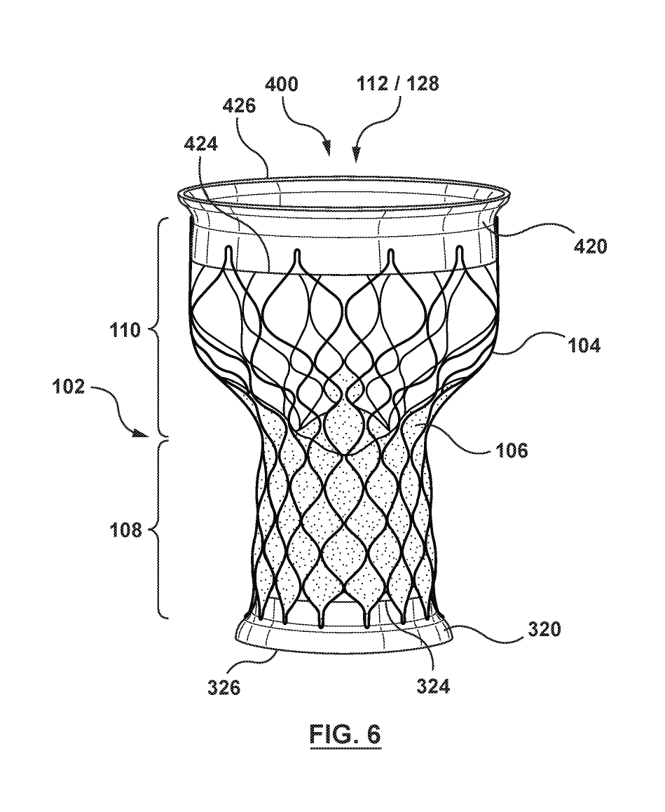

FIG. 6 shows a sealing system 400 according to another embodiment hereof. Sealing system 400 includes a valve component 102 including a frame 104 and a prosthetic valve 106, as described above with respect to FIGS. 2A-2B. Sealing system 400 also includes an expandable sealing ring 320 disposed partially within inflow portion 108 of valve component 102 and partially longitudinally outside of valve component 102, as described above and shown in FIG. 6A. Sealing system 400 further includes a second expandable sealing ring 420 disposed partially longitudinally within outflow portion 110 of valve component 102 and partially longitudinally outside of valve component 102, as shown in FIG. 6. Thus, a longitudinal first end 424 of expandable sealing ring 420 is disposed within outflow portion 110 of valve component 102, and a longitudinal second end 426 of expandable sealing ring 420 is disposed longitudinally downstream of outflow portion 1110.

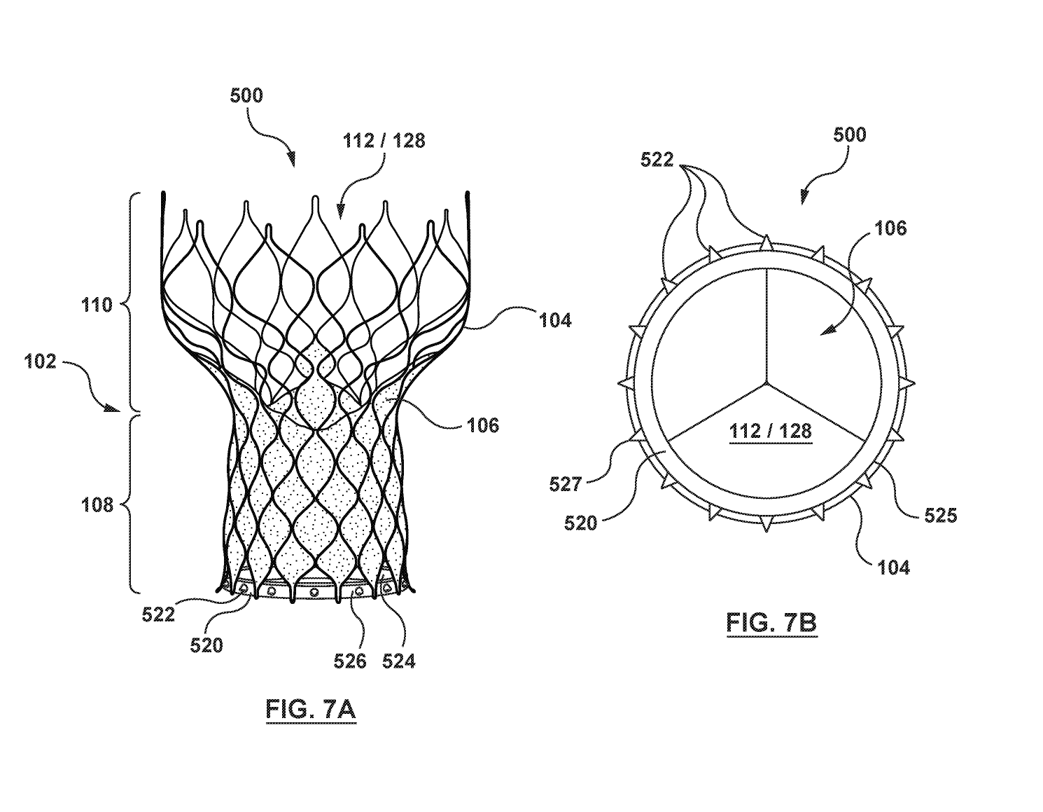

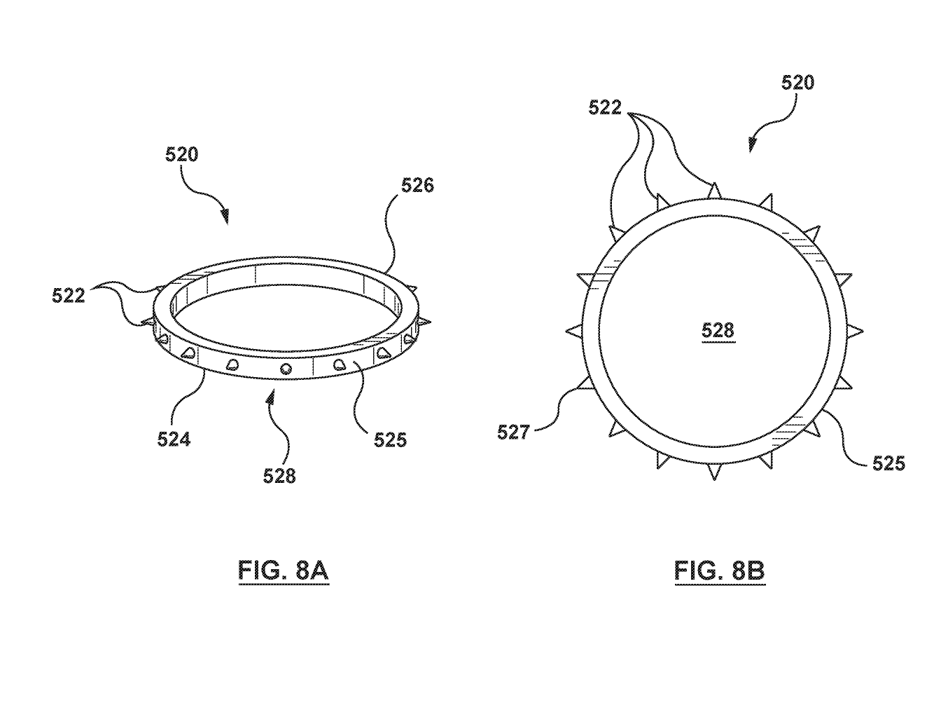

FIGS. 7A, 7B, 8A, and 8B show another embodiment of a sealing system 500. Sealing system 500 includes a valve component 102 and an expandable sealing ring 520. Valve component 102 includes a frame 104 and a prosthetic valve 106, as described above.

Expandable sealing ring 520 is similar to expandable sealing ring 120 described above. Accordingly, expandable sealing ring 500 is a generally annular ring defining a passage 528, as shown in FIGS. 8A-8B. Expandable sealing ring 520 has a longitudinal first end 524 and a longitudinal second end 526. Expandable sealing ring 520 further includes a plurality of protrusions 522 extending radially outward from an outer surface 525 of expandable sealing ring 520, as shown in FIGS. 7B, 8A, and 8B. Protrusions 522 may be formed as contiguous, integral components of expandable sealing ring 520, or may be coupled to expandable sealing ring 520 by methods such as, but not limited to, laser or ultrasonic welding, adhesives, or other methods suitable for the purposes disclosed herein. Protrusions 522 may include a sharp tip 527. Protrusions 522 may be configured such that with valve component 102 in its radially expanded deployed configuration, and expandable sealing ring 520 in an expanded configuration within valve component 102, protrusions 522 extend radially outward from outer surface 525 of expandable sealing ring 520 through frame 104 of valve component 102, and into a wall of a native valve. While a specific number and configuration of protrusions 522 are shown in FIGS. 7A, 7B, 8A, and 8B, this is not meant to limit the design and more or fewer protrusions 522 in various configurations may be utilized.

FIG. 7A shows expandable sealing ring 520 deployed entirely within valve component 102 at inflow portion 108 of valve component 102. However, as explained above, expandable sealing ring 520 may alternatively by deployed at outflow portion 110 of valve component 102, or at both inflow portion 108 and outflow portion 110. Further, expandable sealing ring 520 may be deployed entirely longitudinally within or only partially longitudinally within valve component 102, as described above.

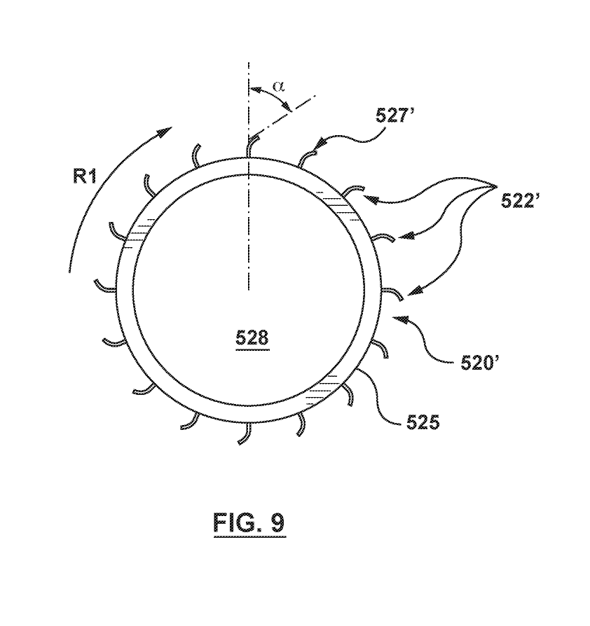

FIG. 9 shows another embodiment of an expandable sealing ring 520′ similar to expandable sealing ring 520. Expandable sealing ring 520′ differs from expandable sealing ring 520 in that protrusions 522′ of expandable sealing ring 520′ extend at an angle α relative to the radial direction. Angle α may be in the range of 15 to 50 degrees relative to the radial direction. Thus, when expandable sealing ring 520′ is disposed within inflow portion 108 or outflow portion 110 of valve component 102, and is rotated in a direction R1, as shown in FIG. 9, protrusions 522′ rotate in direction R1 and engage valve component 102 and the wall of the native valve.

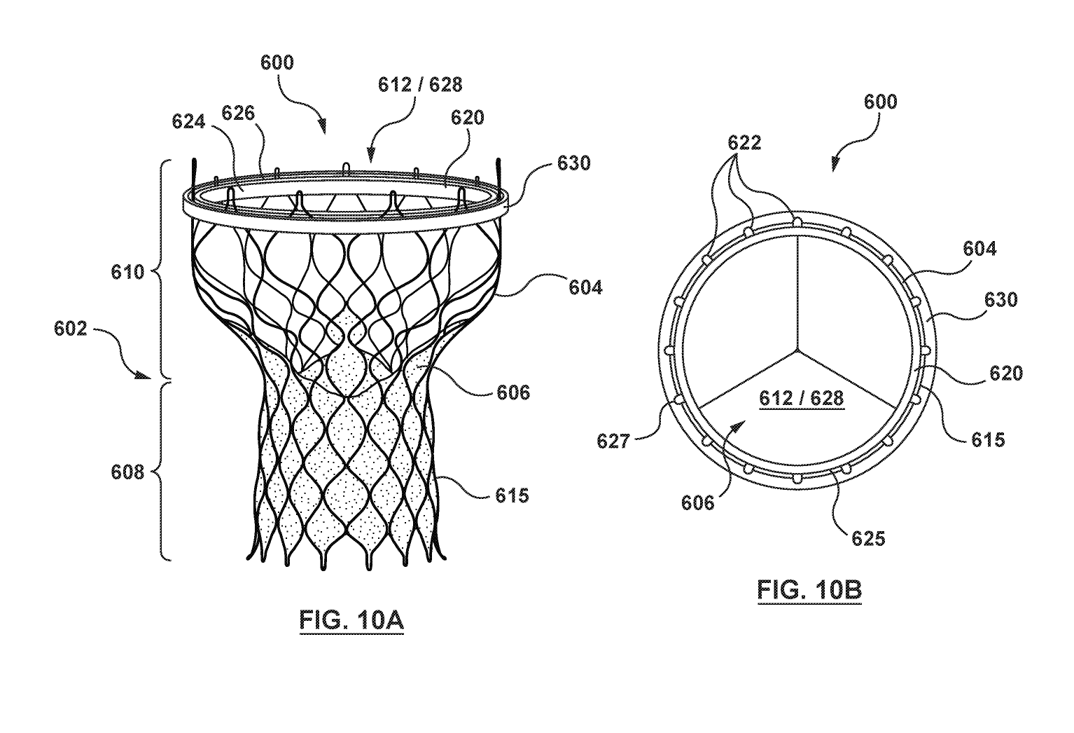

FIGS. 10A-10B show a sealing system 600 in accordance with another embodiment hereof. Sealing system 600 includes a valve component 602, an expandable sealing ring 620, and an outer ring 630. Valve component 602 is similar to the valve component 102 described above, incorporated into this embodiment by reference, and therefore will not be described in detail here. As with valve component 102, valve component 602 includes a frame 604 defining a central passage 612, and a prosthetic valve 606 coupled to frame 604 and disposed within central passage 612. Valve component 602 has an inflow portion 608 at a proximal end of valve component 602, and an outflow portion 610 at a distal end of valve component 602.

Expandable sealing ring 620 of the embodiment of FIGS. 10A-10B is the same as sealing ring 520 described above with respect to FIGS. 7A, 7B, 8A, and 8B. Accordingly, expandable sealing ring 620 is a generally annular ring defining a passage 628. Expandable sealing ring 620 has a longitudinal first end 624 and a longitudinal second end 626, and includes a plurality of protrusions 622 extending radially outward from an outer surface 625 of expandable sealing ring 620, as shown in FIG. 10B, and described above with respect to FIGS. 8A-8B. Protrusions 622 may include a sharp tip 627.

Outer ring 630 is a generally annular ring coupled to an outer surface 615 of valve component 602. Outer ring 630 is deployed with valve component 602. Outer ring 630 and valve component 602 are configured such that outer ring 630 is disposed between frame 604 and a wall of the native valve when valve component 602 is in the radially expanded deployed configuration. Outer ring 630 may be constructed of materials such as, but not limited to polyethylene terephthalate (PET), tissue (including porcine or bovine pericardium), or other biocompatible materials or other materials suitable for the purposes described herein. Outer ring 630 may be secured to frame 604 by methods such as, but not limited to, adhesives, sutures, laser or ultrasonic welding, or any other methods suitable for the purposes described herein.

In the embodiment shown in FIG. 10A, outer ring 630 is deployed radially outside of outflow portion 610 of valve component 602. Further, expandable sealing ring 620 is deployed radially inside of outflow portion 610 and is aligned with outer ring 630. Accordingly, when expandable sealing ring 620 is deployed (i.e., expanded radially outwardly) protrusions 622 extend through frame 604 at outflow portion 610 and into outer ring 630, as shown in FIG. 10B. Although FIGS. 10A-10B show a single expandable sealing ring 620 and a single outer ring 630 disposed at outflow portion 610 of valve component 602, expandable sealing ring 620 and outer ring 630 could alternatively be disposed at inflow portion 608, or there may be multiple expandable sealing rings 620 and outer rings 630 disposed at inflow portion 608, outflow portion 610, or both. Further, both expandable sealing ring 620 and outer ring 630 are shown in FIGS. 10A-10B as being disposed entirely longitudinally between ends of frame 604 (i.e., longitudinally within frame 604). However, expandable sealing ring 620 and outer ring 620 may be disposed partially between ends of frame 604 and partially longitudinally beyond or outside the ends of frame 604, as described above with respect to FIGS. 5A-5B and 6.

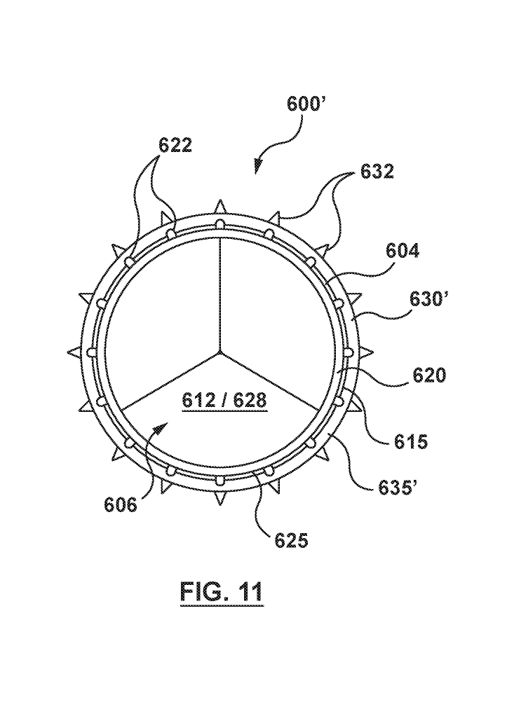

In another embodiment of a sealing system 600′, shown in FIG. 11, an outer ring 630′ may also include a plurality of protrusions 632 extending radially outward from an outer surface 635 of outer ring 630′. Protrusions 632 may be formed as a contiguous, integral component of outer ring 630′, or may be coupled to outer ring 630′ by methods such as, but not limited to laser or ultrasonic welding, adhesives, or other methods suitable for the purposes disclosed herein. Protrusions 632 may be configured such that protrusions 632 extend radially outward from outer surface 635 of outer ring 630′ and into the wall of the native valve. While a specific number and configuration of protrusions 632 are shown in FIG. 11, this is not meant to limit the design and more or fewer protrusions 632 in various configurations are envisioned based upon the application. Other details of sealing system 600′ of FIG. 11 are the same as sealing system 600 of FIGS. 10A-10B, and therefore are not described with respect to FIG. 11

While the various embodiments shown and described with respect to FIGS. 2A-11 provide possible configurations for sealing systems consistent with systems, devices, and methods of the present disclosure, they are not meant to limit the sealing systems to these configurations, and other materials, shapes, and combinations of expandable sealing rings and outer rings may be utilized. Further, each feature of each embodiment shown and/or described can be used in combination with the features of any other embodiment.

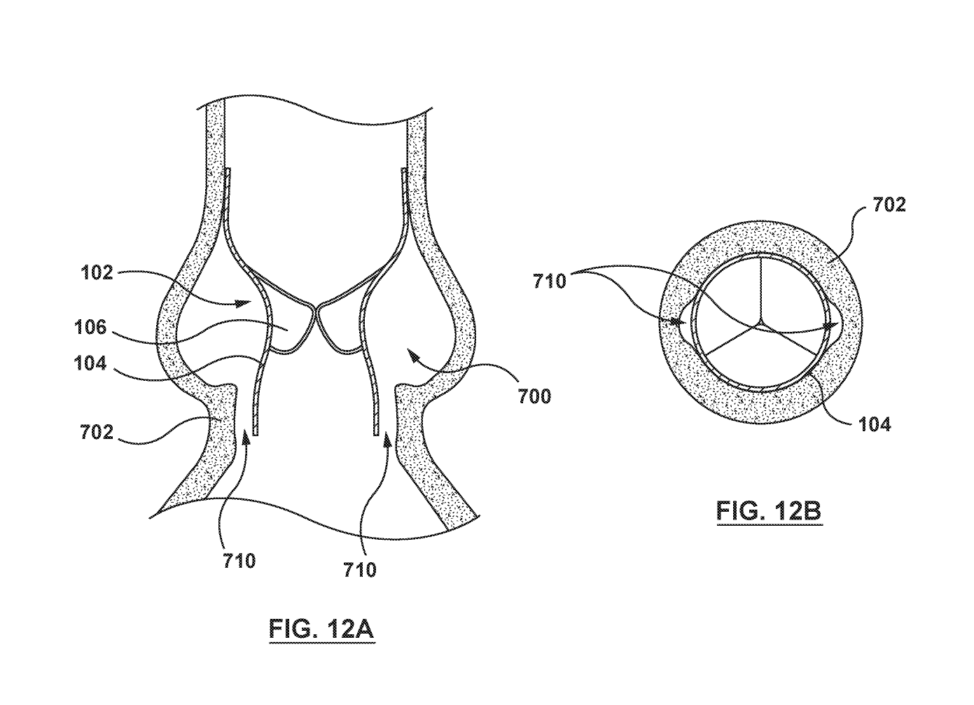

FIGS. 12A-14B schematically show an embodiment of a method of sealing a valve component to a wall of a native valve. The method of FIGS. 12A-14B can also be referred to as a method of remodeling an already deployed valve component or valvular prosthesis. FIGS. 12A-14B show the method using valve component 102, including frame 104 and prosthetic valve 106, and expandable sealing ring 120. However, this is merely exemplary, and the valve components and expandable sealing rings of other embodiments may be utilized. Further, in the embodiment of the method shown, expandable sealing ring 120 is disposed at inflow portion 108 of valve component 102. However, expandable sealing ring 120 may be disposed at outflow portion 110, or additional expandable sealing rings may be utilized and deployed at both inflow portion 108 and outflow portion 110, as described above.

FIGS. 12A-12B shows valve component 102 after it has been delivered and deployed at the site of a native valve 700. Methods and devices for delivering and deploying valve component 102 are known. Whether at the time of deployment or thereafter, and due to various factors, such as the misshapen nature or heavy calcification of walls of the native valve, valve component 102 is not 100% coapted to the wall of the native valve 700. As a result, voids 710 are present, which may result in paravalvular leakage (PVL).

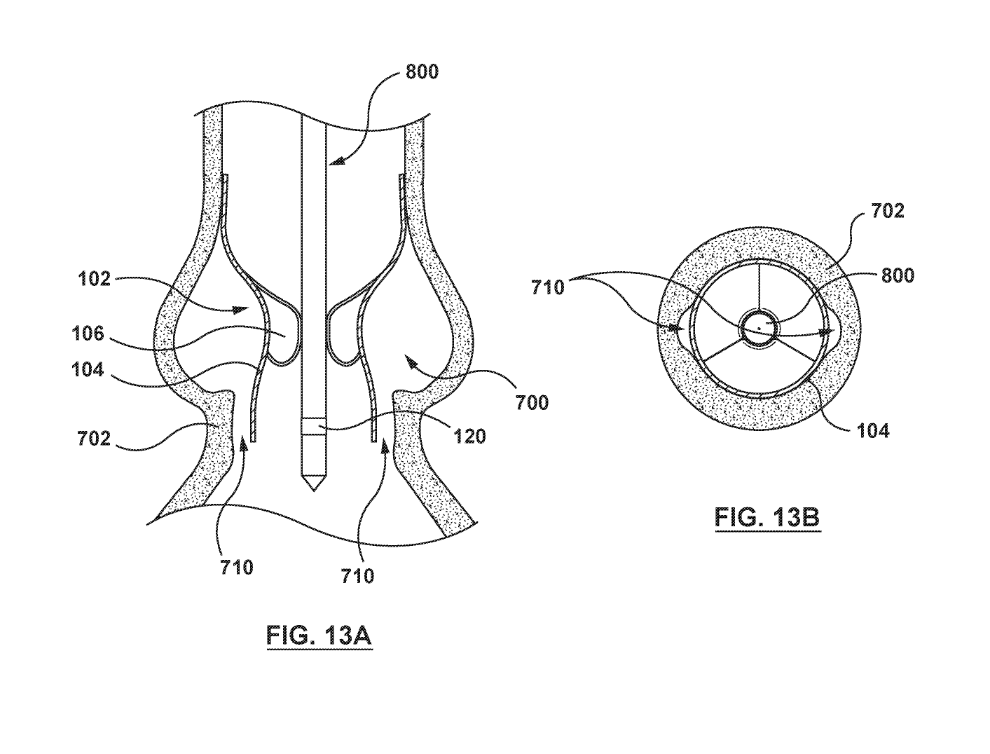

A delivery device 800 with a sealing ring 120 in a radially compressed configuration therein, is advanced through the patient's vasculature and is positioned within valve component 102, with valve component 102 in a radially expanded configuration, using established percutaneous transcatheter procedures, as shown in FIGS. 13A-13B.

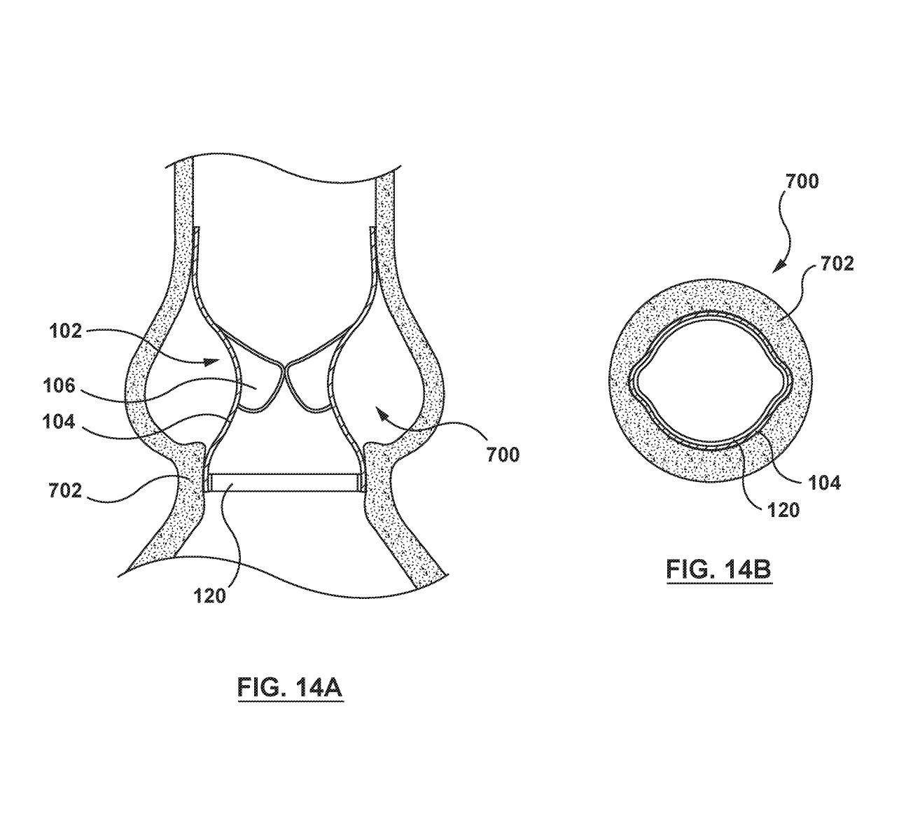

Expandable sealing ring 120 is deployed from delivery device 800 using known percutaneous transcatheter procedures, as shown in FIGS. 14A-14B. For example, and not by way of limitation, if expandable sealing ring 120 is self-expanding, expandable sealing ring 120 may be radially compressed in a sheath of delivery system 800 for delivery to the native valve 700. Once at the desired location, the sheath is retracted proximally, thereby enabling expandable sealing ring 120 to self-expand to its natural or pre-set expanded configuration. As expandable sealing ring 120 radially expands, expandable sealing ring 120 forces frame 104 of valve component 102 against the wall of the native valve 700, as shown in FIGS. 14A-14B. In the embodiment shown, the wall of the native valve is the aortic annulus 702 because the native valve is the aortic valve and the paravalvular leakage was determined to be cause at the inflow portion of valve component 102. However, expandable sealing ring 120 may be disposed in other portions of valve component 102 such that expansion of sealing ring 120 forces frame 104 against other walls of the native valve, as explained above. Further, if the expandable sealing ring 120 is balloon expandable or otherwise mechanically expandable, expandable sealing ring may be mounted on a balloon of a delivery system or coupled to a mechanical expansion mechanism. When the delivery system is at the desired location, the balloon or mechanical expansion mechanism is expanded, thereby expanding expandable sealing ring 120.

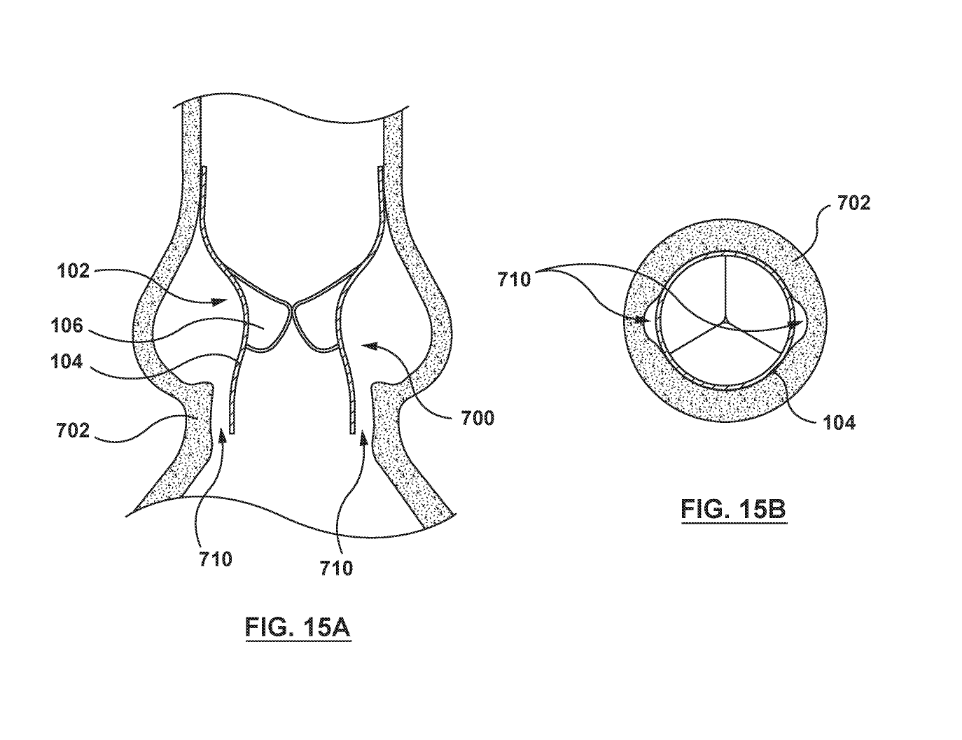

FIGS. 15A-17B show another embodiment of a method of sealing a valve component to a wall of a native valve. The method of FIGS. 15A-17B can also be referred to as a method of remodeling an already deployed valve component or valvular prosthesis. FIGS. 15A-17B show the method using expandable sealing ring 520 and valve component 102 of FIGS. 7A-8B. However, other embodiments as described above, in particular expandable sealing ring 520′ of FIG. 9, may also be used. Further, in the embodiment of the method shown, expandable sealing ring 520 is disposed at inflow portion 108 of valve component 102. However, expandable sealing ring 520 may be disposed at outflow portion 110, or additional sealing rings may be utilized and deployed at both inflow portion 108 and outflow portion 110, as described above.

FIGS. 15A-15B show valve component 102 after it has been delivered and deployed at the site of a native valve 700. Methods and devices for delivering and deploying valve component 102 are known. Whether at the time of deployment or thereafter, and due to various factors, such as the misshapen nature or heavy calcification of walls of the native valve, valve component 102 is not 100% coapted to the wall of the native valve 700. As a result, voids 710 are present, which may result in paravalvular leakage (PVL)

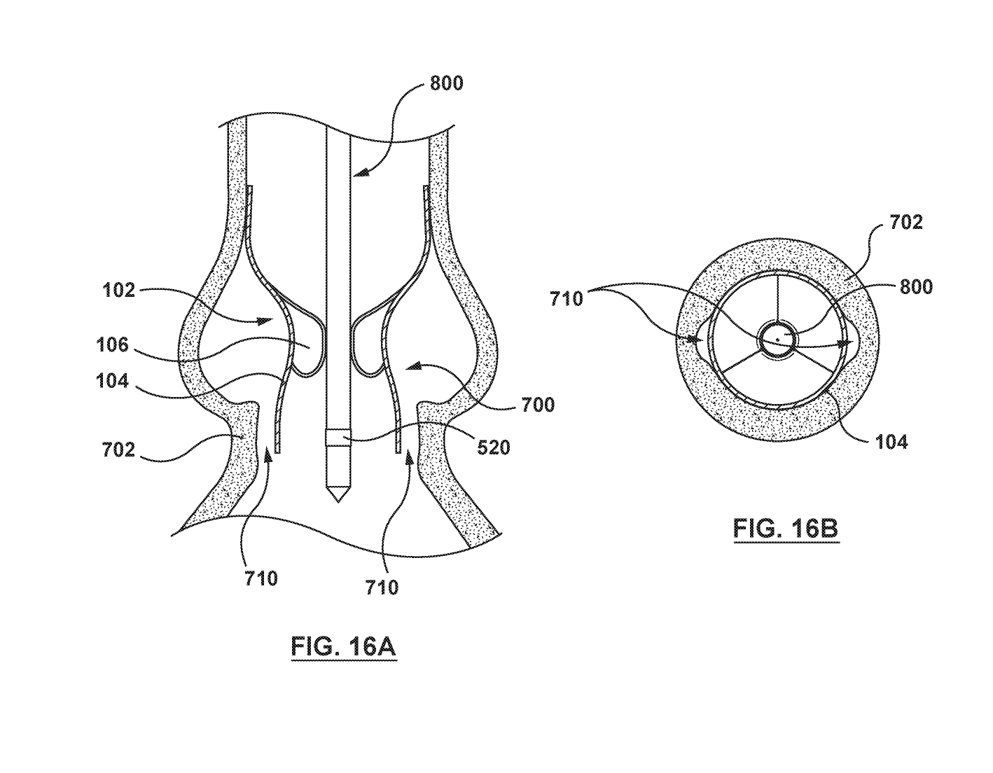

As shown in FIG. 16A-16B, a delivery device 800 with a sealing ring 520 in a radially compressed configuration therein, is advanced through the patient's vasculature and positioned within valve component 102, with valve component 102 in a radially expanded configuration, using established percutaneous transcatheter procedures

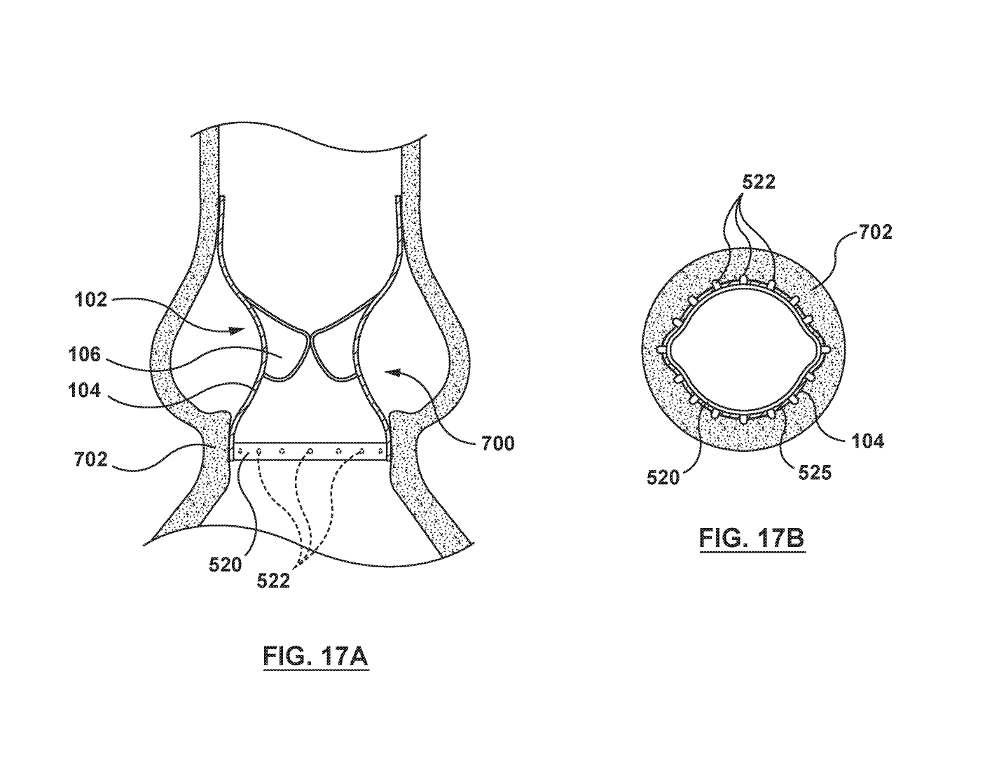

Expandable sealing ring 520 is deployed from delivery device 800 using known percutaneous transcatheter procedures, as shown in FIGS. 17A-17B. For example, and not by way of limitation, if expandable sealing ring 520 is self-expanding, expandable sealing ring 520 may be radially compressed in a sheath of delivery system 800 for delivery to the native valve 700. Once at the desired location, the sheath is retracted proximally, thereby enabling expandable sealing ring 520 to self-expand to its natural or pre-set expanded configuration. As expandable sealing ring 520 radially expands, expandable sealing ring 520 forces frame 104 of valve component 102 against the wall of the native valve 700, as shown in FIGS. 17A-17B. Further, as explained above, expandable sealing ring 520 includes protrusions 522 extending radially outward from outer surface 525. Therefore, as expandable sealing ring 520 radially expands, protrusions 522 extend through frame 104 of valve component 102 and engage the wall of the native valve 700, as shown in FIGS. 17A-17B.

In the embodiment shown, the wall of the native valve is the aortic annulus 702 because the native valve is the aortic valve and the paravalvular leakage was determined to be caused at the inflow portion of valve component 102. However, expandable sealing ring 520 may be disposed in other portions of valve component 102 such that expansion of sealing ring 520 forces valve component 102 against other walls of the native valve, as explained above. Further, if the expandable sealing ring 520 is balloon expandable or otherwise mechanically expandable, expandable sealing ring 520 may be mounted on a balloon of a delivery system or coupled to a mechanical expansion mechanism. When the delivery system is at the desired location, the balloon or mechanical expansion mechanism is expanded, thereby expanding expandable sealing ring 520.

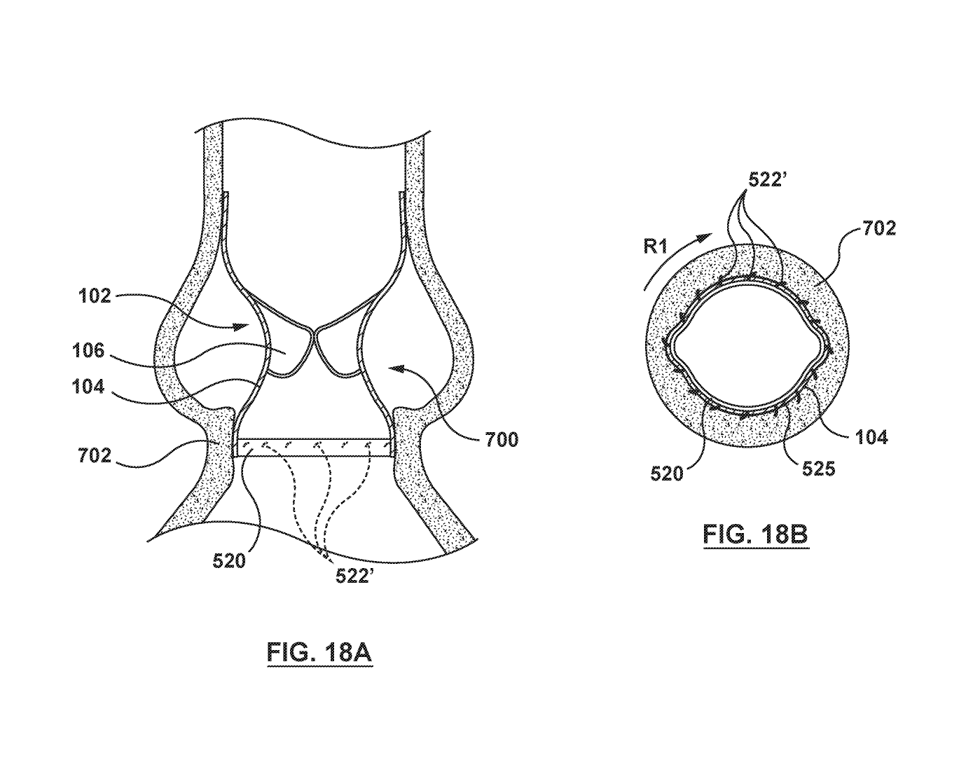

In another embodiment of the method, expandable sealing ring 520′ is utilized, with the plurality of protrusions 522′ which extend radially outward at an angle α relative to the radial direction. In this method, after sealing ring 520′ is expanded radially outward to the radially expanded configuration, sealing ring 520′ is rotated in a direction R1 such that protrusions 522′ engage valve component 102 and the wall of the native valve 700, as shown in FIGS. 18A-18B.

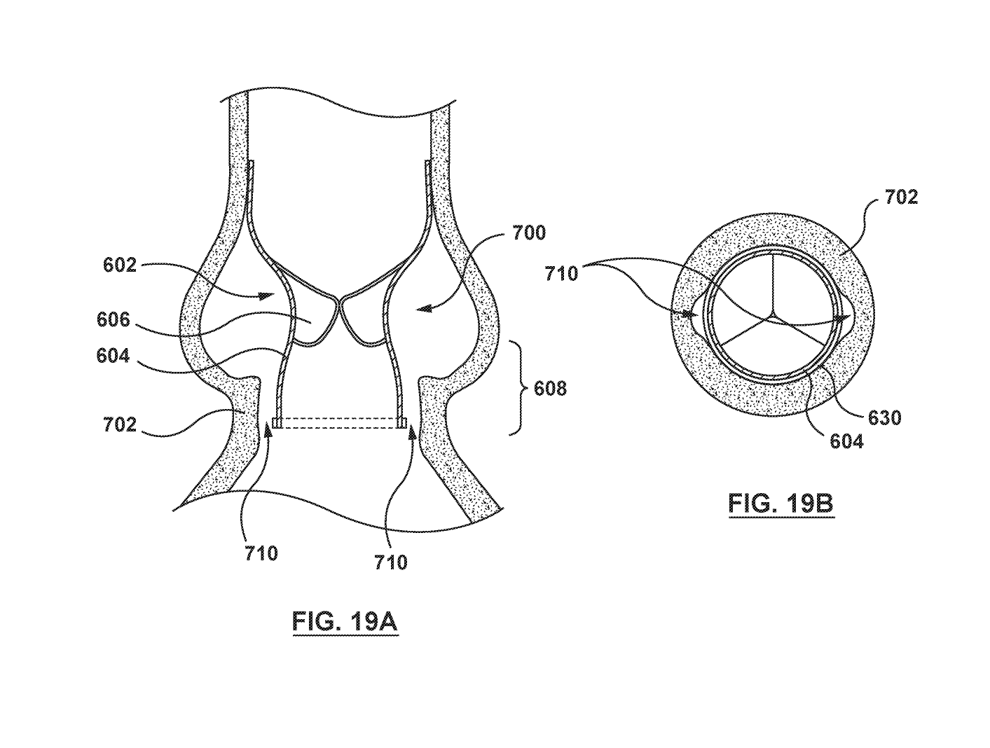

FIGS. 19A-21B schematically show an embodiment of a method of sealing a valve component to a wall of a native valve utilizing the sealing system 600 of FIGS. 10A-10B. The method of FIGS. 19A-21B can also be referred to as a method of remodeling an already deployed valve component or valvular prosthesis. FIGS. 19A-19B show valve component 602, including frame 604, prosthetic valve 606, and outer ring 630, after it has been delivered and deployed at the site of a native valve 700. Outer ring 630 is disposed between the wall of the native valve 700 and frame 604 with frame 604 in a radially expanded configuration. Methods and devices for delivering and deploying valve component 602 are known. Whether at the time of deployment or thereafter, and due to various factors, such as the misshapen nature or heavy calcification of the wall of the native valve 700, valve component 602 is not 100% coapted to wall of the native valve 700. As a result, voids 710 are present, which may result in paravalvular leakage (PVL).

A delivery device 800 with a sealing ring 620 in a radially compressed configuration is advanced through the patient's vasculature and positioned within frame 604, with frame 604 in the radially expanded configuration, using known percutaneous transcatheter procedures. Sealing ring 620 includes a plurality of protrusions 622 extending radially outward from an outer surface 625 of sealing ring 620. Delivery device 800 is advanced within frame 604 such that sealing ring 620 is aligned with outer ring 630.

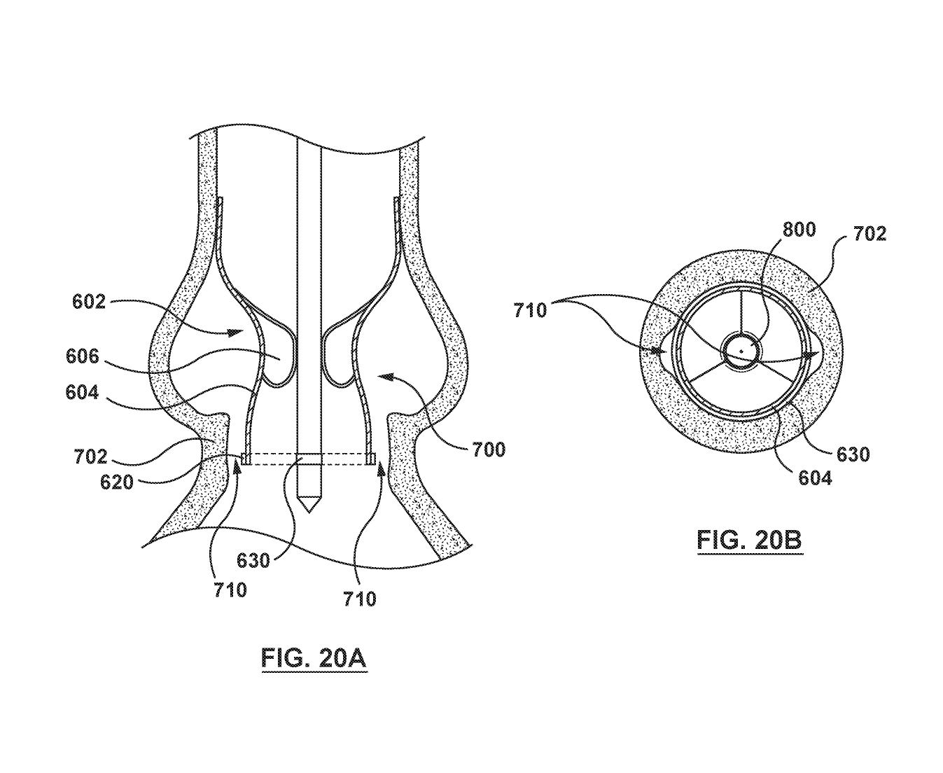

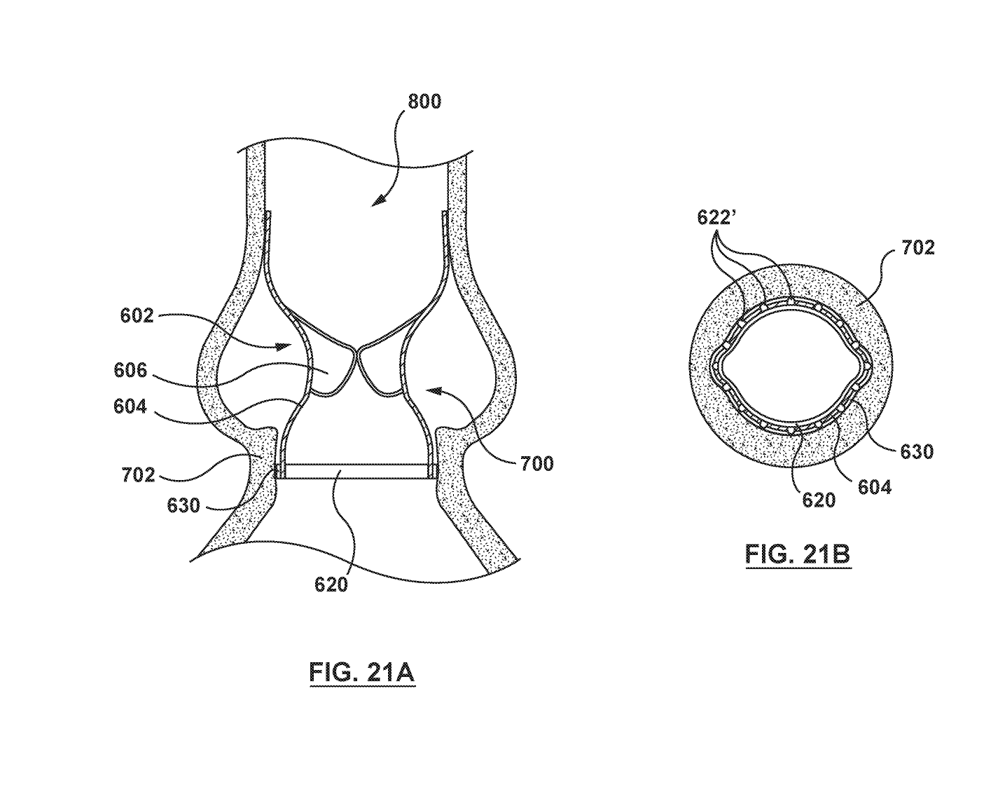

Expandable sealing ring 620 is deployed from delivery device 800 using known percutaneous transcatheter procedures, as shown in FIG. 20A-20B. For example, and not by way of limitation, if expandable sealing ring 620 is self-expanding, expandable sealing ring 620 may be radially compressed in a sheath of delivery system 800 for delivery to the native valve 700. Once at the desired location, the sheath is retracted proximally, thereby enabling expandable sealing ring 620 to self-expand to its natural or pre-set expanded configuration. As expandable sealing ring 520 radially expands, expandable sealing ring 620 forces frame 604 of valve component 602 radially outward towards the wall of the native valve 700. Further, protrusions 622 of sealing ring 620 expand radially outward with sealing ring 620 and into outer ring 630, as shown in FIGS. 21A-21 B.

In the method shown in FIGS. 19A-21B, outer ring 630 is shown without projections. The method described in FIGS. 19A-21B may also be used with the outer ring 630′ described with respect to FIG. 11. Using outer ring 630′, when expandable sealing ring 620 expanded radially outwardly, expandable sealing ring forces fame 604 and outer ring 630′ radially outwardly, and protrusions 632 of outer ring 630′ are forced radially outward into the wall of the native valve 700, as shown in FIG. 11.

Similar methods as previously described may be used for various embodiments and configurations of the present disclosure including, but not limited to a plurality of sealing rings, various configurations of protrusions on sealing rings and outer rings, and varied positioning of sealing rings and outer rings at both inflow and/or outflow portions of the valve component, as described herein.

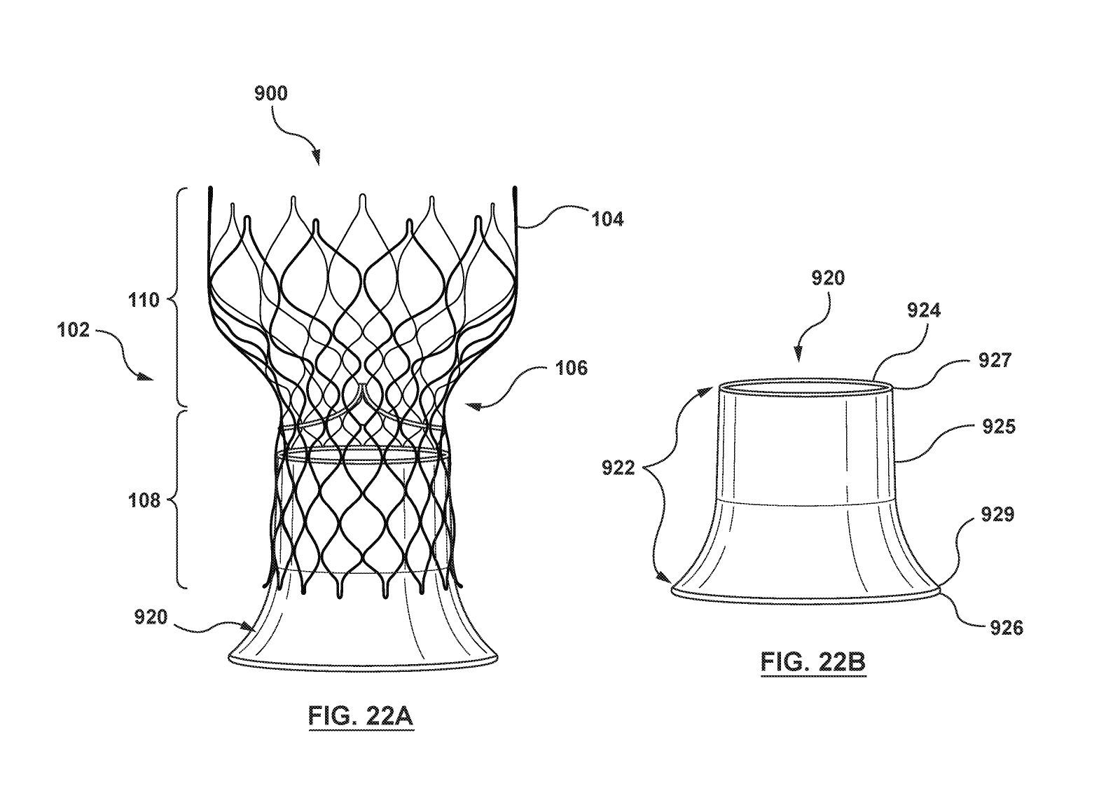

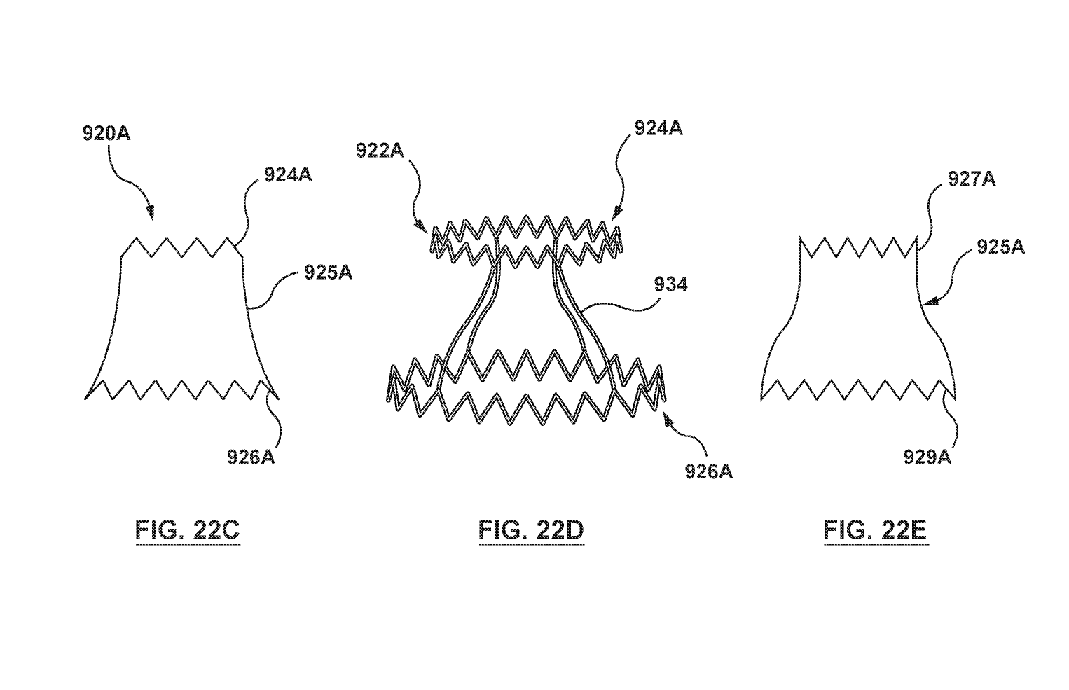

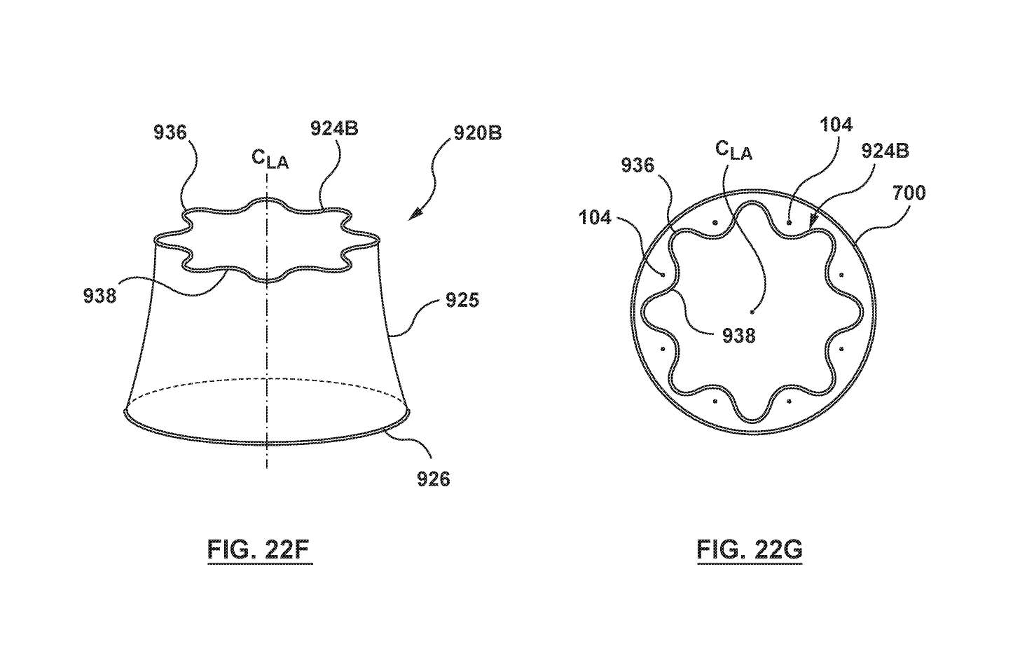

FIGS. 22A-22C illustrate another embodiment of a sealing system 900 in accordance with the present disclosure. The sealing system 900 includes a valve component 102 and an expandable sealing component 920. In FIG. 22A, valve component 102 is in a radially expanded configuration.

Valve component 102 includes a frame 104 and a prosthetic valve 106, as shown in FIG. 22A. Details of valve component 102, including frame 104 and prosthetic valve 106 are described above. Therefore, details of valve component 102 will not be repeated with respect to the present embodiment.

In an embodiment, expandable sealing component 920 is of a generally tubular shape and includes a sealing frame 922 and a skirt 925. Sealing component 920 may also be referred to as a device for preventing blood flow radially between valve component 102 and a wall of the native valve complex, as will be described in more detail below. In an embodiment, sealing frame 922 includes a first ring 924 disposed at a first longitudinal end of sealing component 920 and a second ring 926 disposed at a second longitudinal end of sealing component 920 opposite first ring 924, as shown in FIG. 22B. Skirt 925 includes a first end 927 coupled to first ring 924, and a second end 929 coupled to second ring 926, as shown in FIGS. 22B. Expandable sealing component 920 has a radially compressed configuration for delivery to a treatment site and a radially expanded configuration when deployed. Expandable sealing component 920 may be self-expanding, balloon expandable, or otherwise mechanically expandable.

In an example where the expandable sealing component is used at the site of a native aortic valve, the diameter of first ring 924 of expandable sealing component 920 in the radially expanded deployed configuration may be in the range of 16 to 30 millimeters. In such an example, the diameter of second ring 926 in the radially expanded deployed configuration may be in the range of 20 to 40 millimeters. In some embodiments, the diameter of second ring 926 is larger than the diameter of the first ring 924 such that expandable sealing component 920 is generally frustoconical in shape. However, it is recognized that first ring 924 and second ring 926 of expandable sealing component 920 may have a smaller or larger respective expanded diameters depending on the application. Further, the unrestrained expanded diameter of first ring 924 and second ring 926 of expandable sealing component 920 may be about 2-6 millimeters larger than the diameter of each respective location in which first ring 924 and second ring 926 is to be installed. This is to create opposing radial forces between the outward radial forces of expandable sealing component 920 against inward resisting forces of the wall of the native valve/valve complex and/or valve component 902. First ring 924 and second ring 926 of expandable sealing component 920 may be constructed of materials such as, but not limited to stainless steel, Nitinol, cobalt-chromium alloys (e.g., L605), nickel-cobalt-chromium alloys (e.g., MP35N®) or other materials suitable for the purposes described herein. Skirt 925 of expandable sealing component 920 may be constructed of materials such as, but not limited to polyester, nylon, expanded polytetrafuoroethyline (ePTFE), natural tissue, or other materials suitable for the purposes described herein. Skirt 925 may be coupled to first ring 924 and second ring 926 by methods such as, but not limited to adhesives, sutures, or other methods suitable for the purposes described herein.

In the embodiment of FIG. 22A, expandable sealing component 920 is deployed at an inflow portion 108 of valve component 102. First ring 924 of expandable sealing component 920 is disposed within inflow portion 108, proximal (upstream) of the leaflets of prosthetic valve 106. Second ring 926 is disposed within the native valve complex (not shown in FIG. 22A), proximal (upstream) of inflow portion 108. Skirt 925 extends from second ring 926 to first ring 924, as shown in FIG. 22A. Skirt 925 covers (blocks) PVL passages 710 (FIG. 1), preventing blood flow into PVL passages 710 (FIG. 1). Skirt 925 further directs blood flow to inflow portion 108 of valve component 102. First ring 924 may also push frame 104 of inflow portion 108 radially outward to close off PVL passages 710, as described above. However, using sealing component 920, flow is directed into valve component 102 even if first ring 924 does not sufficiently seal frame 104 against the wall of the native valve complex. Although not shown in FIGS. 22A-22B, expandable sealing component 920 may alternatively be disposed at an outflow portion 110 of valve component 102 in some applications.

First ring 924 and second ring 926 of sealing frame 922 are shown as solid rings. However, they are not so limited. FIGS. 22C-2D show an alternative first ring 924A and second ring 926A. As can be seen, first and second rings 924A, 926A are undulating, or zig-zag structures including peaks, valleys, and struts connecting the peaks and the valleys. In some embodiments, first and second rings 924A, 926A may be generally sinusoidal. In an embodiment shown in FIG. 22C and 22E, a first end 927A and a second end 929A of a skirt 925A may be undulating to match the peaks and valleys of first ring 924A and second ring 926A.

FIG. 22D shows another alternative embodiment of sealing frame 922A, wherein longitudinal connectors 934 couple first ring 924A and second ring 924B to each other. Longitudinal connectors 934 may be similar to longitudinal connectors 334 described above with respect to FIG. 5B. Further, longitudinal connections 934 are not limited to the embodiment of FIG. 22D. Instead, they may be included with other embodiments described herein.

Further, although two types of rings for the sealing frame have been described, this disclosure is not so limited, and other types of rings may be utilized. Further, different types of rings may be used for the first ring and the second ring. Still further, the skirt may be attached to an inner surface of an outer surface of the sealing frame.

FIGS. 22F-22G show another embodiment of a sealing component 920B including a sealing frame including a first ring 924B and a second ring 926, and a skirt 925. In the embodiment of FIGS. 22F-22G, first ring 924B is profiled or shaped to engage spaces between the wire members of frame 104 of valve component 102. In the embodiment shown, first ring 924B undulates toward and away from a central longitudinal axis C_{LA} of sealing component 920B. Thus, first ring 924B includes portions 936 extending away from the central longitudinal axis C_{LA} and portions 938 extending towards the central longitudinal axis C_{LA}. As can be seen in FIG. 22G, when sealing component 920B is implanted within valve component 102, portions 936 extend between the wire members of frame 104 towards a wall of the native valve complex 700, as shown in FIG. 22G. This provides a secure connection between first ring 924B and frame 104 of valve component 102.

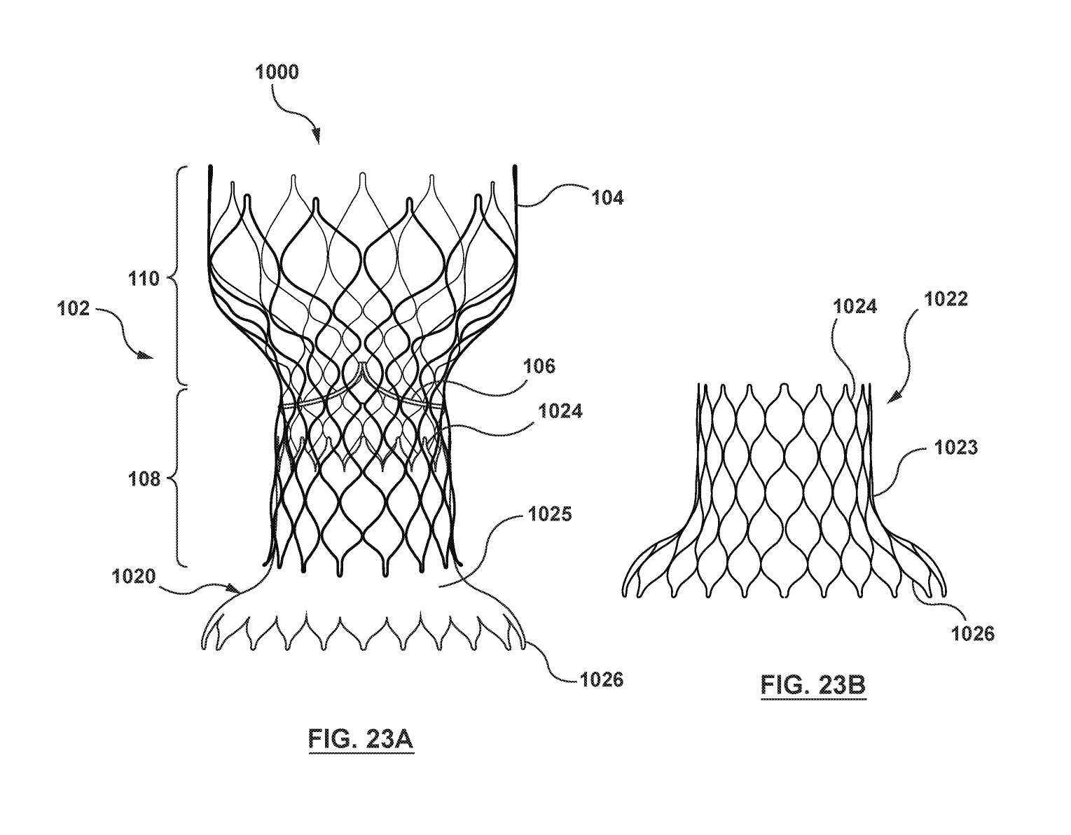

FIGS. 23A-23B show a sealing system 1000 according to another embodiment hereof. Sealing system 1000 includes valve component 102 including frame 104 and prosthetic valve 106, as shown in FIG. 23A and described above. Sealing system 1000 further includes an expandable sealing component 1020.

In an embodiment, expandable sealing component 1020 is of a generally tubular shape including a sealing frame 1022 and a skirt 1025 coupled to the sealing from 1022. In the embodiment shown in FIGS. 23A-23B, sealing frame 1022 is a support structure that comprises a number of struts or wire portions arranged relative to each other to provide a desired compressibility and strength for the purposes described herein. Sealing frame 1022 may be self-expandable, balloon-expandable, or otherwise mechanically expandable. Sealing frame includes a first end 1024 (which may also be referred to as a ring), a second end 1026 (which may also be referred to as a ring) opposite first end 1024, and a body portion 1023 between first end 1024 and second end 1026, as shown in FIG. 23B. Skirt 1025 may be coupled to an inner surface or an outer surface of sealing frame 1022. Materials for sealing frame 1033 and skirt 1025 may be as described above with respect to expandable sealing component 920.

In an embodiment shown in FIG. 23A, expandable sealing component 1020 is deployed at an inflow portion 108 of valve component 102. First end 1024 of expandable sealing component 1020 is disposed within inflow portion 108, proximal of the leaflets of prosthetic valve 106. Second end 1026 is disposed within the native valve complex (not shown in FIG. 23A), proximal (upstream) of inflow portion 108. Skirt 1025 extends between first end 1024 and second end 1026, as shown in FIG. 23A. Skirt 1025 covers (blocks) PVL passages 710 (FIG. 1), preventing blood flow into PVL passages 710 (FIG. 1). Skirt 1025 further directs blood flow to inflow portion 108 of valve component 102. Sealing frame 1022 may also push frame 104 of inflow portion 108 radially outward to close off PVL passages 710, as described above. However, using sealing component 1020, flow is directed into valve component 102 even if sealing frame 1020 does not sufficiently seal frame 104 against the wall of the native valve complex. Although not shown in FIGS. 23A-23B, expandable sealing component 1020 may alternatively be disposed at an outflow portion 110 of valve component 102.

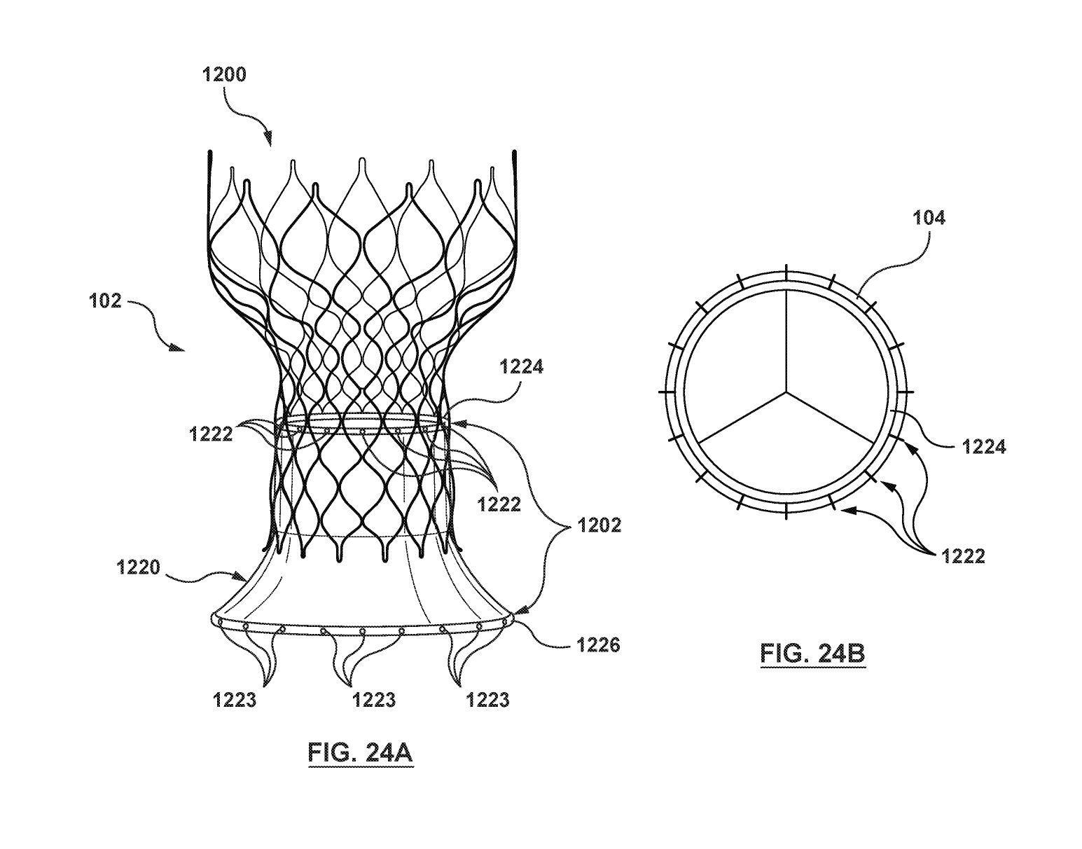

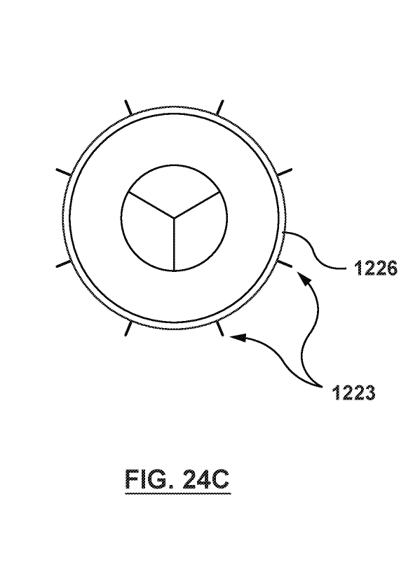

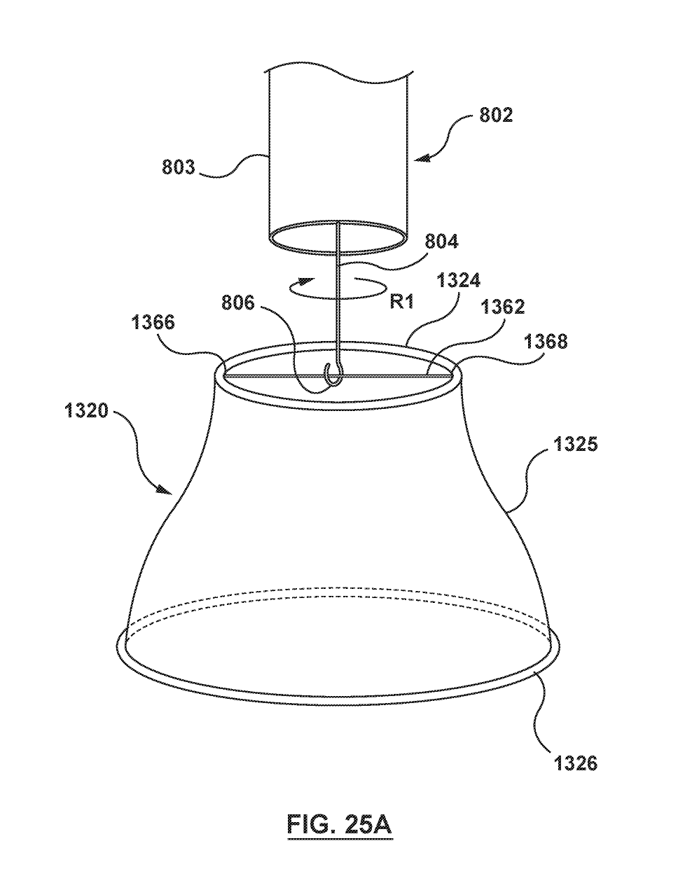

FIGS. 24A-24C show a sealing system 1200 according to another embodiment hereof. Sealing system 1200 includes valve component 102 and an expandable sealing component 1220.

Expandable sealing component 1220 is similar to expandable sealing component 920 described above. Therefore, all of the details and alternatives of expandable sealing component 1220 will not be repeated. However, generally, expandable sealing component includes an expandable sealing frame 1202 and a skirt 1225 coupled to the sealing frame 1202. In an embodiment, sealing frame 1202 includes a first ring 1224 and a second ring 1226, as described above. First ring 1224 includes a plurality of first protrusions 1222, and second ring 1226 includes a plurality of second protrusions 1223, as shown in FIG. 24A. Each first protrusion 1222 and each second protrusion 1223 extend radially outward from an outer surface of first ring 1224 and second ring 1226, respectively, as shown in FIGS. 24B-24C, respectively. Each first protrusion 1222 and each second protrusion 1223 may be formed as a contiguous, integral component of first ring 1224 and second ring 1226, respectively. Alternatively, each first protrusion 1222 and each second protrusion 1223 may be coupled to first ring 1224 and second ring 1226, respectively, by methods such as, but not limited to laser or ultrasonic welding, adhesives, or other methods suitable for the purposes disclosed herein.

The plurality of first protrusions 1222 are configured such that with valve component 102 in an expanded configuration and first ring 1224 in an expanded configuration and disposed within valve component 102, the plurality of first protrusions 1222 extend radially outward and extend through frame 104, as shown in FIG. 24B, and into a wall of a native valve complex. Alternately, the plurality of first protrusions 1222 may be formed as hooks, tines, or other shapes suitable for the purposes described herein and configured to couple first ring 1224 to the wall of the native valve 700 (not shown in FIGS. 24A-24B) and/or frame 104 of valve component 102.

The plurality of second protrusions 1223 are configured such that with second ring 1226 (in an expanded configuration) within the native valve complex, the plurality of second protrusions 1223 extend radially outward, as shown in FIG. 24C, and into a wall of the native valve complex. Alternately, the plurality of second protrusions 1223 may be formed as hooks, tines, or other shapes suitable for the purposes described herein.

While a specific number and configuration of the plurality of first and second protrusions 1222 and 1223 are shown in FIGS. 24A-24C, this is not meant to limit the design and more or fewer first or second protrusions 1222 and 1223 in various configurations may be utilized. Moreover, the presence of the plurality of first protrusions 1222 on first ring 1224 does not necessitate the presence of the plurality of second protrusions 1223 on second ring 1226. Likewise, the presence of the plurality of second protrusions 1223 on second ring 12126 does not mandate the plurality of first protrusions 1222 on first ring 1224. Still further, while the plurality of first protrusions and second protrusions are described with respect to an embodiment of the sealing frame having a first ring and a second ring similar to FIGS. 22A-22B, this is not meant to be limiting. Thus, radially outward extending protrusions may be added to any of the embodiments described herein.

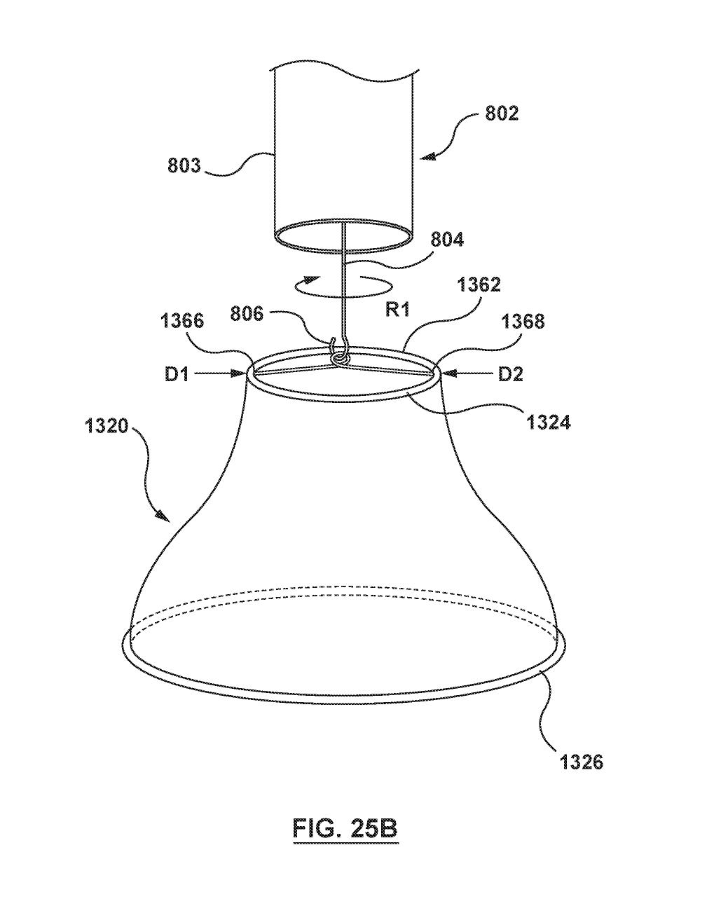

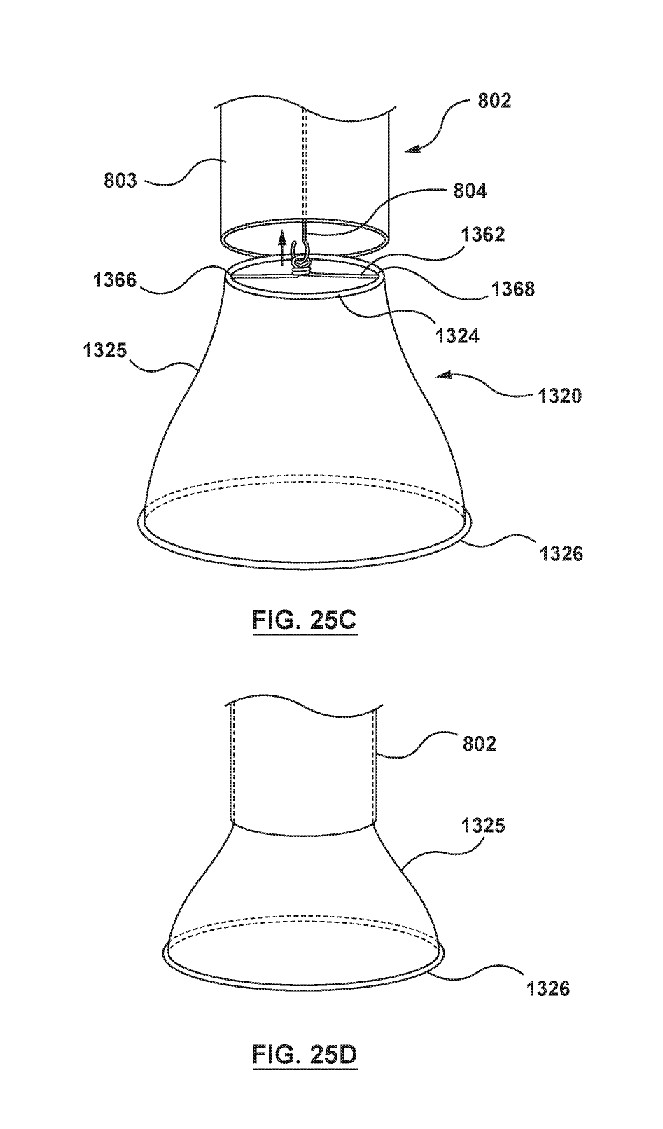

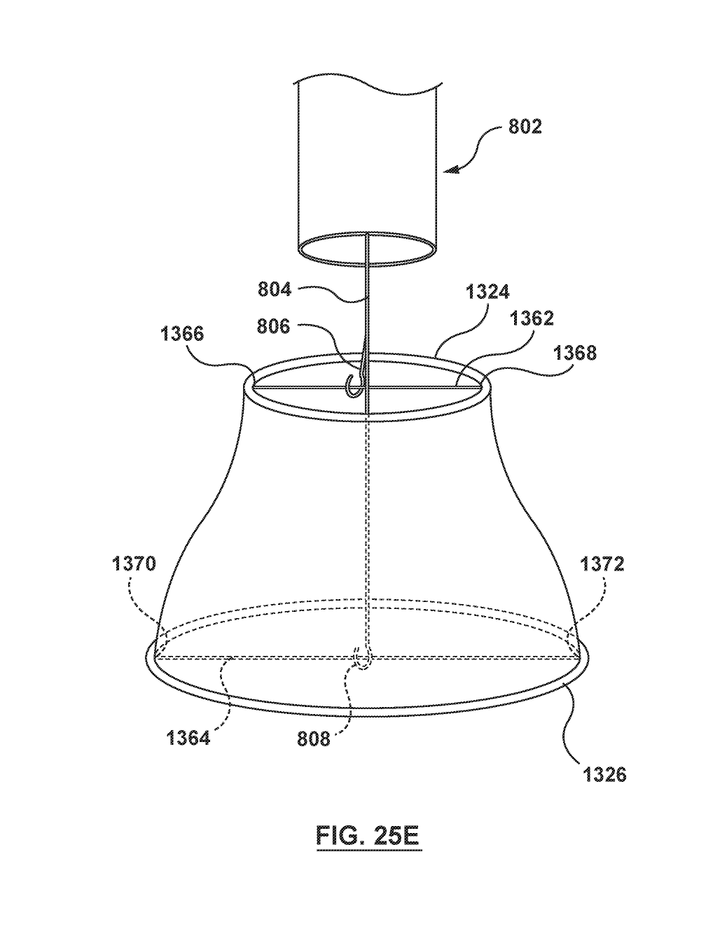

FIGS. 25A-25D show an expandable sealing component 1320 of a sealing system according to another embodiment hereof. Expandable sealing component 1320 as shown in FIGS. 25A-25D is similar to expandable sealing component 920 shown in FIGS. 22A-22B, except that expandable sealing component 1320 is retrievable. Accordingly, expandable sealing component includes a sealing frame including a first ring 1324 and a second ring 1326. Expandable sealing component further includes a skirt 1325 coupled to first ring 1324 and second ring 1326, as described above. However, expandable sealing component 1320 may be similar to any other of the embodiments described herein, with the added retrievability described below.

In an embodiment, expandable sealing component 1320 includes a first cross-member 1362 extending across first ring 1324, as shown in FIG. 25A. First cross-member 1362 is an elongate element, filament, wire, or groups thereof, and is not limited to a particular cross-sectional shape or material. First cross-member 1262 are configured such that expandable sealing component 1320 may be collapsed for retrieval/removal from the sealing system, as described in greater detail below. Expandable sealing component 1320 may be retrieved/removed in situations where expandable sealing component 1320 may have assisted in remodeling the native valve complex to resolve the paravalvular leakage, tissue ingrowth into the valve component resolved the paravalvular leakage, a second valve component needs to be implanted within the first valve component, or other reasons why expandable sealing component 1320 may need to be or desired to be retrieved.

First cross-member 1362 includes a first end 1366 coupled to first ring 1324, and a second end 1368 coupled to an opposite side of first ring 1324, as shown in FIG. 25A. Thus, first cross-member 1362 extends in a radial direction across an opening of first ring 1324 (i.e., across the diameter of second ring 1326). First cross-member 1362 is configured such that pulling or twisting first cross-member 1362 causes first ring 1324 to radially collapse.

In an embodiment shown in FIGS. 25A-25D, a retrieval device 802 includes a tube 803 and a snare device 804 include a hook 806. Snare device 804 extends through tube 803 and out of a distal end of tube 803. Snare device 804 is extended until hook 806 snags, grasps, or otherwise snares first cross-member 1362, as shown in FIG. 25A.

With first cross-member 1362 snared by hook 806, snare device 804 may be rotated as shown by arrows R1 in FIGS. 25A and 25B. By rotating snare device 804 as it is engaged with first cross-member 1362, first cross-member 1362 wraps around snare device 804. As the first cross-member 1362 wraps around snare device 804, first end 1366 moves in a first direction D1 and second end 1368 moves in a second direction D2, as shown in FIG. 25B. Thus, first end 1366 and second end 1368 move towards each other. Moreover, as first end 1366 and second end 1368 draw closer together, first ring 1324 of expandable sealing component 1320 radially collapses.

When first ring 1324 has radially collapsed such that its diameter is smaller than the inner diameter of tube 803, snare device 804 is retracted towards tube 803, as shown in FIG. 25C. As snare device 804 continues to move expandable sealing component 1320 within tube 803, tube 803 forces second ring 1326 to radially collapse.

In another embodiment shown in FIG. 25E, a second cross-member 1364 may extend across second ring 1326. Thus, second cross-member 1364 includes a first end 1370 coupled to second ring 1326, and a second end 1372 coupled to an opposite side of second ring 1326. Thus, second cross-member 1364 extends in a radial direction across an opening of second ring 1326 (i.e., across the diameter of second ring 1326). In such an embodiment, snare device 804 may include a second hook 808 to snag, grasp, or otherwise snare second cross-member 1364. Thus, when snare device 804 is rotated, as described above, both first cross-member 1362 and second cross-member 1364 wrap around snare device 804, thereby collapsing first ring 1324 and second ring 1326, as described above.

First cross-member 1362 and second cross-member 1364 may be constructed of materials such as, but not limited to stainless steel, Nitinol, nylon, polybutester, polypropylene, silk, polyester, or other materials suitable for the purposes described herein. First cross-member 1362 and second cross-member 1364 may be coupled to first ring 1324 and second ring 1326, respectively by methods such as, but not limited to laser or ultrasonic welding, adhesives, tying, or other methods suitable for the purposes described. First cross-member 1362 and second cross-member 1364 may further include loops, hooks, or other devices configured to facilitate snagging, grasping, or snaring by retrieval device 802, as would be understood by those skilled in the pertinent art. Alternatively, the snare device 804 may pull/push or otherwise manipulate first cross-member 1362 and/or second cross-member 1364 to radially collapse first ring 1324 and second ring 1326, respectively.

Although FIGS. 25A-25E show expandable sealing component 1320 retrievable by manipulation of first or first and second cross-members 1362, 1364, this is not meant to limit the design, and other methods of retrieval or configurations may be utilized. Examples include, but are not limited to, devices and methods for retrieval described in U.S. patent application Ser. No. 15/008,019 filed Jan. 27, 2016, which is incorporated by reference herein in its entirety.

As explained above, retrievable expandable sealing component 1320 is configured to be retrieved after implantation for various possible reasons. However, if sealing component 1320 is to be retrieved, tissue ingrowth during its time at the treatment site may hinder the ability to retrieve sealing component 1320. Accordingly, in some embodiments, expandable sealing component 1320 may be configured to inhibit tissue ingrowth. For example, and not by way of limitation, the surface finish of the sealing component 1320 may be smooth and non-porous to inhibit tissue ingrowth. In another example, the skirt 1325 may include a positively charged coating. In other embodiments, the sealing component may include a biologically or pharmacologically active substance to inhibit tissue ingrowth. The biologically or pharmacologically active substance to inhibit tissue ingrowth may be applied as a coating to expandable sealing component 1320, such as a coating on skirt 1325. The biologically or pharmacologically active substance to inhibit tissue ingrowth may be, for example and not by way of limitation, a hydrophobic coating, a biocompatible, non-biodegradable polymer such as silicon rubber (polydimethyldiloxane), polyurethane, collagen gel or other appropriate form of biocompatible polymers.

In the embodiments where the sealing component is not retrievable, it may be desirable to promote tissue ingrowth to secure the sealing component at the desired location. Therefore, the sealing component may be configured to promote tissue ingrowth. For example, and not by way of limitation, the skirt may be textured or may be knit or woven into a porous fabric. Larger pore sizes tend to promote tissue ingrowth. In other examples, the sealing component may include a biologically or pharmacologically active substance to promote tissue ingrowth. The biologically or pharmacologically active substance to promote tissue ingrowth may be applied as a coating to the expandable sealing component, such as a coating to the skirt. The biologically or pharmacologically active substance to promote tissue ingrowth may be, for example and not by way of limitation, fibrin and growth factors.

While the various embodiments shown and described provide possible configurations for sealing systems consistent with systems, devices, and methods of the present disclosure, they are not meant to limit the sealing systems to these configurations, and other materials, shapes, and combinations of expandable sealing components may be utilized. Further, each feature of each embodiment shown and/or described can be used in combination with the features of any other embodiment.

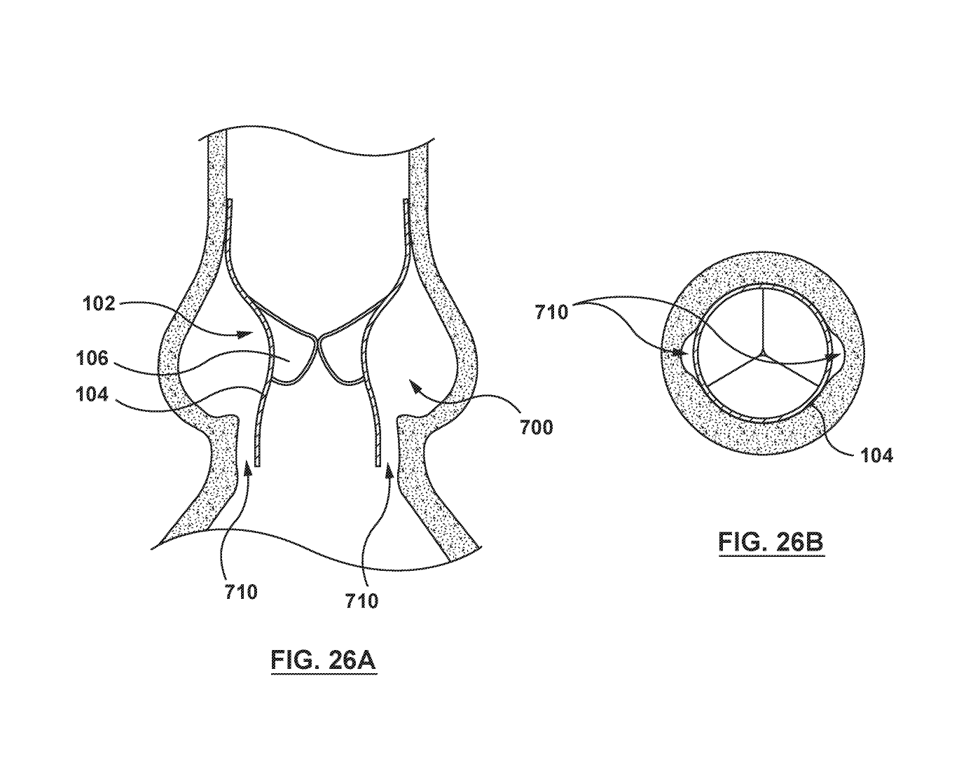

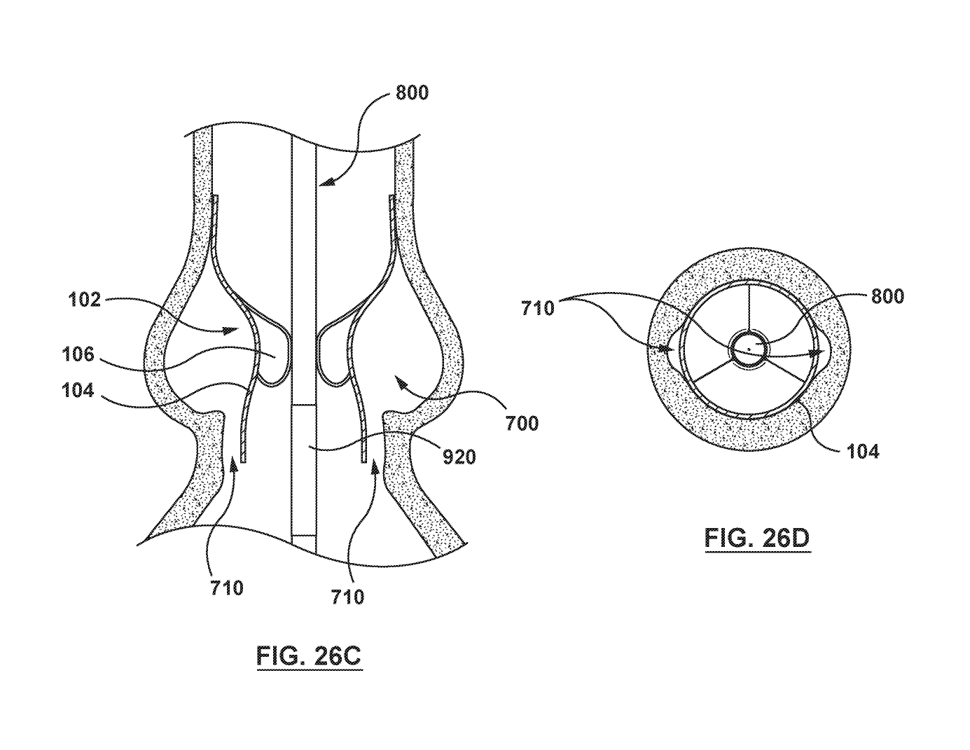

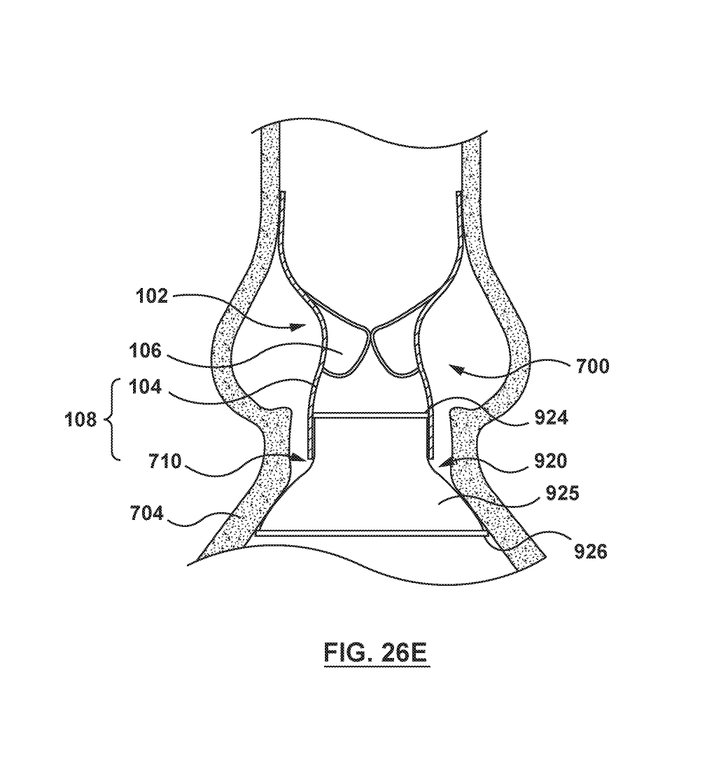

FIGS. 26A-26E schematically show an embodiment of a method of sealing a valve component to a wall at the site of a native valve. FIGS. 26A-26E show the method using valve component 102, including frame 104 and prosthetic valve 106, and expandable sealing component 920. However, this is merely exemplary, and the valve components and expandable sealing components of other embodiments may be utilized. Further, in the embodiment of the method shown, expandable sealing component 920 is disposed at inflow portion 108 of valve component 102. However, expandable sealing component 920 may be disposed at outflow portion 110, or additional expandable sealing components may be utilized and deployed at both inflow portion 108 and outflow portion 110.

FIGS. 26A-26B show valve component 102 after it has been delivered and deployed at the site of a native valve 700. Methods and devices for delivering and deploying valve component 102 will be understood by those skilled in the art. After deployment of valve component 102, and due to various factors, such as the misshapen nature or heavy calcification of walls of the native valve 700, valve component 102 may not be 100% coapted to the wall of the native valve 700. As a result, voids 710 are present, which may result in paravalvular leakage (PVL).

Using established percutaneous transcatheter procedures a delivery device 800 with an expandable sealing component 920 in a radially compressed configuration therein, is advanced through the patient's vasculature. The delivery device is positioned so that expandable sealing component 920 is positioned partially within inflow portion 108 of valve component 102 and partially longitudinally upstream of with valve component 102, as shown in FIGS. 26C-26D.

Expandable sealing component 920 is deployed from delivery device 800 using known percutaneous transcatheter procedures, as shown in FIG. 26E. For example, and not by way of limitation, if expandable sealing component 920 is self-expanding, expandable sealing component 920 may be radially compressed in a sheath of delivery system 800 for delivery to the native valve 700. Once at the desired location, the sheath is retracted proximally, thereby enabling expandable sealing component 920 to self-expand to its natural or pre-set expanded configuration. In another example, if expandable sealing component 920 is balloon expandable, expandable sealing component is mounted over a balloon of delivery system 800. Once at the desired location, the balloon is inflated, thereby radially expanding expandable sealing component 920. As expandable sealing component 920 radially expands, first ring 924 of expandable sealing component 920 expands and contacts frame 104 of valve component 102. Generally simultaneously, second ring 926 of expandable sealing component 920 radially expands against a wall 704 of the native valve complex, as shown in FIG. 26E. First ring 924 contacts frame 104 within inflow portion 108 and below leaflets of prosthetic valve 106. Second ring 926 contacts the wall 704 of the native valve complex longitudinally outside the inflow portion 108 of the valve component 102 (upstream in this embodiment). Skirt 925, disposed between second ring 926 and first ring 924 covers PVL passages 710, preventing blood flow to PVL passages 710 and directs blood flow to inflow portion 108 of valve component 102.

The devices and methods described above have been shown with respect to a native aortic valve. However, this is not meant to be limiting, and the devices and methods described herein may be used in other valves of the heart, such as the mitral and tricuspid valves.

While only some embodiments and methods have been described herein, it should be understood that it has been presented by way of illustration and example only, and not limitation. Various changes in form and detail can be made therein without departing from the spirit and scope of the invention, and each feature of each embodiment discussed herein, and of each reference cited herein, can be used in combination with the features of any other embodiment. All patents and publications discussed herein are incorporated by reference herein in their entirety.

What is claimed is:

1. A sealing system comprising:
a valve component including a frame and a prosthetic valve coupled to the frame, the valve component having a radially expanded deployed configuration, an inflow portion and an outflow portion, the frame defining a central passage, the prosthetic valve disposed within the central passage of the frame; and
an expandable sealing component including a sealing frame and a skirt coupled to the frame, the expandable sealing component configured to be inserted in a radially compressed configuration within the central passage with the valve component in the radially expanded deployed configuration, the expandable sealing component further configured to be radially expanded to a radially expanded configuration partially within the valve component such that a first portion of the sealing component is disposed within the valve component and a second portion of the sealing component is disposed longitudinally outside the valve component, the expandable sealing component configured to prevent blood flow radially between the valve component and a wall of the native valve complex.

2. The sealing system of claim 1, wherein the first portion is disposed within the inflow portion of the valve component, the second portion is disposed longitudinally outside the inflow component in an upstream direction, and the expandable sealing component is configured to direct blood flow into the inflow portion of the valve component.

3. The sealing system of claim 1, wherein the first portion includes a first ring, the second portion includes a second ring, a first end of the skirt is coupled to the first ring, and a second end of the skirt is coupled to the second ring.

4. The sealing system of claim 3, wherein the first ring of the expandable sealing component is shaped and configured to extend into spaces between members of the frame of the valve component when the expandable sealing component is in the radially expanded configuration.

5. The sealing system of claim 4, wherein the first ring undulates toward and away from a central longitudinal axis of the sealing component.

6. The sealing system of claim 3, wherein the first ring of the expandable sealing component further comprises a plurality of protrusions, extending radially outwardly.

7. The sealing system of claim 6, wherein the plurality of protrusions of the first ring are configured to couple the first ring of the expandable sealing component to a wall of the valve component when the expandable sealing component is in the radially expanded configuration.

8. The sealing system of claim 3, wherein the second ring of the expandable sealing component comprises a plurality of protrusions extending radially outwardly.

9. The sealing system of claim 8, wherein the plurality of protrusions of the second ring are configured to embed in the wall of the native valve complex when the expandable sealing component is in the radially expanded configuration.

10. The sealing system of claim 1, wherein the expandable sealing component is configured to be retrievable.

11. The sealing system of claim 10, wherein the expandable sealing component is configured to inhibit tissue ingrowth.

12. The sealing system of claim 1, wherein the expandable sealing component is configured to promote tissue ingrowth.

13. The sealing system of claim 1, where in the expandable sealing component is selected from the group consisting of self-expanding, balloon expandable, and mechanically expandable.

14. A method of correcting paravalvular leakage of an installed valvular prosthesis, the valvular prosthesis including a frame and a prosthetic valve coupled to the frame, the method comprising the steps of:
advancing an expandable sealing component in a radially compressed configuration to a location partially within a portion of the frame of the valvular prosthesis with the frame in a radially expanded configuration within a native valve complex, the expandable sealing component including a sealing frame and a skirt coupled to the sealing frame; and
expanding the expandable sealing component to a radially expanded configuration such that the expandable sealing component prevents blood flow radially between the valvular prosthesis and a wall of the native valve complex.

15. The method of claim 14, wherein the step of advancing the expandable sealing component comprises advancing the expandable sealing component to a location such that a first portion of the expandable sealing component is disposed in the inflow portion of the valvular prosthesis, and a second portion of the sealing component is disposed longitudinally outside the inflow portion of the valvular prosthesis in an upstream direction such that the expandable sealing component directs blood flow into the inflow portion.

16. The method of claim 15, wherein the first portion includes a first ring, the second portion includes a second ring, a first end of the skirt is coupled to the first ring, and a second end of the skirt is coupled to the second ring.

17. The method of claim 16, wherein the first ring includes a plurality of protrusions, and wherein the step of expanding the expandable sealing component includes forcing the plurality of protrusions radially outward such that the plurality of protrusions of the first ring engage a wall of the valvular prosthesis.

18. The method of claim 16, wherein the second ring includes a plurality of protrusions, and wherein the step of expanding the expandable sealing component includes forcing the plurality of protrusions radially outward into the native wall of the native valve complex.

19. The method of claim 14, wherein the step of expanding the expandable sealing component is selected from the group consisting of releasing the expandable sealing component such that the expandable sealing component self-expands, inflating a balloon to expand the expandable sealing component, and actuating a mechanical expansion mechanism to expand the expandable sealing component.
